(19) United States
(12) Patent Application Publication
(10) Pub. No.: US 2023/0165276 A1
(43) Pub. Date: Jun. 1, 2023

(54) NOVEL USE OF SUBSTITUTED 2H-CHROMENS AND THEIR DERIVATIVES

(71) Applicant: DSM IP ASSETS B.V., Heerlen (NL)

(72) Inventors: Laure CLASADONTE, Kaiseraugst (CH); André DUESTERLOH, Kaiseraugst (CH); Weerasinghe INDRASENA, Kaiseraugst (CH); Thomas NETSCHER, Kaiseraugst (CH); René, Tobias STEMMLER, Kaiseraugst (CH); Christof WERHLI, Kaiseraugst (CH)

(21) Appl. No.: 17/041,714

(22) PCT Filed: Mar. 29, 2019
(86) PCT No.: PCT/EP2019/058062

§ 371 Date: Sep. 25, 2020

(30) Foreign Application Priority Data
Mar. 29, 2018 (EP) 18164853.6

(51) Int. Cl. A23K 20/121; C07D 311/76; A23K 20/158; A23K 50/40

(52) U.S. Cl. CPC A23K 20/121; C07D 311/76; A23K 20/158; A23K 50/40

(57) ABSTRACT

The present invention is directed towards the use of 2H-chromens and their derivatives of formula (I) and/or of formula (II) wherein R¹ and R² are independently from each other H or C₁₋₁₁-alkyl or (CH₂)ₙ—OH with n being an integer from 1 to 6 or R¹ and R² together represent a keto group, and wherein R³, R⁴, R⁵, and R⁶ are independently from each other H or C₁₋₆-alkyl or C₁₋₆-alkoxy, and R⁷ is H or C₁₋₆-alkyl, as antioxidants, especially in feed such as pet food and feed ingredients such as fish meal, insect meal and poultry meal, as well as PUFA-containing oil such as marine oil, microbial oil, fungal oil, algal oil and PUFA-containing plant oil. The present invention is further directed towards feed ingredients and feed for insects, aquatic and terrestrial animals comprising such 2H- chromens and their derivatives of formula (I) and/or of formula (II).

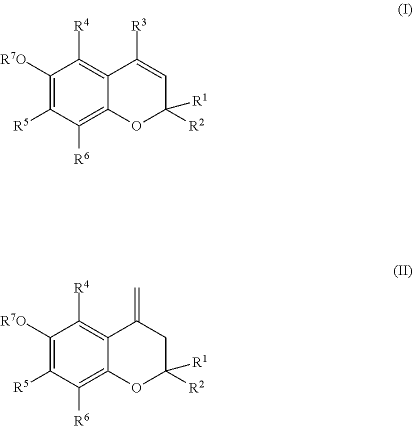

### CROSS-REFERENCE TO RELATED APPLICATIONS

This application is the U.S. national phase of International Application No. PCT/EP2019/058062 filed 29 Mar. 2019, which designated the U.S. and claims priority to EP Patent Application No. 18164853.6 filed 29 Mar. 2018, the entire contents of each of which are hereby incorporated by reference.

### FIELD

The present invention is directed to the use of a compound of formula (I) and/or a compound of formula (II) as antioxidant,

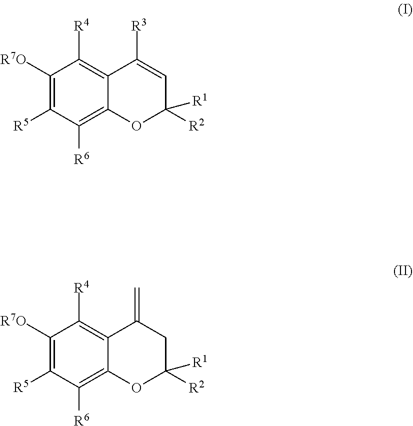

wherein R¹ and R² are independently from each other H or C₁₋₁₁-alkyl or (CH₂)ₙ—OH with n being an integer from 1 to 6 or R¹ and R² together represent a keto group, and

wherein R³, R⁴, R⁵, and R⁶ are independently from each other H or C₁₋₆-alkyl or C₁₋₆-alkoxy, and

R⁷ is H or C₁₋₆-alkyl.

### BACKGROUND AND SUMMARY

The compounds of the present invention are efficient as antioxidants, preferably in feed and feed ingredients. The compounds of the present invention are especially efficient as antioxidants in feed comprising proteins and/or unsaturated fatty acid (derivative)s and in feed ingredients comprising proteins and/or unsaturated fatty acid (derivative)s. “Derivatives” are e.g. the monoglycerides, diglycerides and triglycerides as well as C₁₋₆-alkyl esters such as the methyl and ethyl esters.

Unmodified fish meal can spontaneously combust from heat generated by oxidation of the polyunsaturated fatty acids in the fish meal. In the past, factory ships have sunk because of such fires. Strict rules regarding the safe transport of fish meal have been put in place by authorities and the International Maritime Organization (IMO). According to IMO, fishmeal must be stabilized with antioxidants to prevent spontaneous combustion during overseas transport and storage.

The shipping regulations of the United Nations for the Transport of Dangerous Goods (UN-TDG) currently only allow ethoxyquin and BHT as antioxidants to stabilize fish meal for marine transport. But authorization of ethoxyquin has now been suspended in the European Union due to safety and health concerns.

BHT must be added in higher quantities to achieve the same efficacy as ethoxyquin. Furthermore, BHT is currently under safety evaluation by ECHA and its re-registration as feed additive is pending in Europe.

Therefore, there is a need to replace ethoxyquin and BHT as an antioxidant.

### DETAILED DESCRIPTION

This need is fulfilled by the present invention, which is directed to the use of a compound of formula (I) and/or a compound of formula (II) as antioxidant,

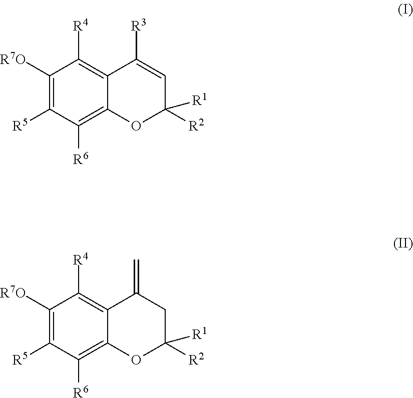

wherein R¹ and R² are independently from each other H or C₁₋₁₁-alkyl or (CH₂)ₙ—OH with n being an integer from 1 to 6 or R¹ and R² together represent a keto group, and

wherein R³, R⁴, R⁵, and R⁶ are independently from each other H or C₁₋₆-alkyl or C₁₋₆-alkoxy, and

R⁷ is H or C₁₋₆-alkyl;

and with the preferences for the substituents R¹ to R⁷ as given below.

“alkyl” and “alkoxy” in the context of the present invention encompass linear alkyl and branched alkyl, and linear alkoxy and branched alkoxy, respectively.

If one of R¹ and R² is an alkyl with more than 4 C-atoms or if one of R¹ and R² is a (CH₂)ₙ—OH group with more than 4 C-atoms, the other one is preferably H.

Preferably R¹ and R² are independently from each other H or C₁₋₄-alkyl or (CH₂)ₙ—OH with n being an integer from 1 to 4, R³, R⁴, R⁶ and R⁷ are independently from each other H or C₁₋₄-alkyl, and R⁵ is H or C₁₋₄-alkyl or C₁₋₄-alkoxy.

More preferably R¹ and R² are independently from each other H or C₁₋₂-alkyl or (CH₂)ₙ—OH with n being 1 or 2, R³, R⁴, R⁶ and R⁷ are independently from each other H or C₁₋₂-alkyl, and R⁵ is H or C₁₋₂-alkyl or C₁₋₂-alkoxy.

Even more preferably R¹ and R² are independently from each other H or methyl or (CH₂)—OH, R³, R⁴, R⁶ and R⁷ are independently from each other H or methyl or ethyl, and R⁵ is H or methyl or methoxy.

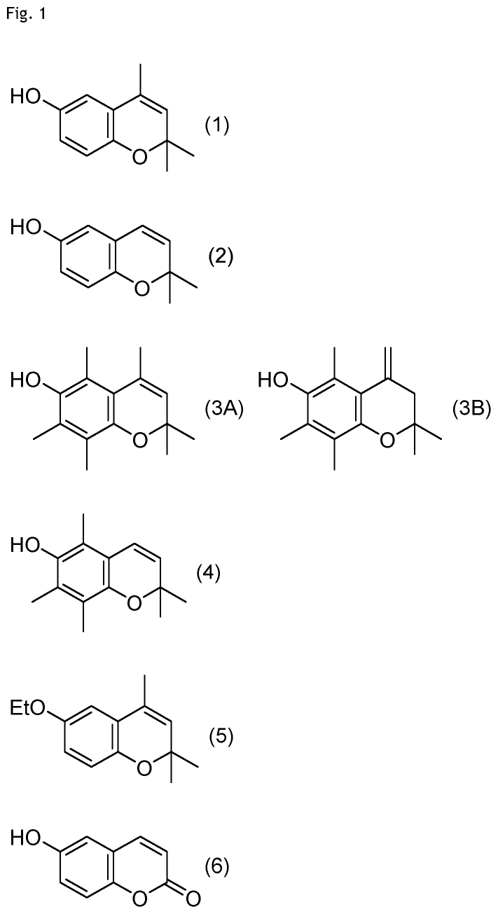

Especially preferred are the following compounds of formulae (1) to (7) (see also FIG. 1), whereby compounds of formulae (1) to (5) are preferred and compound of formula (1) is most preferred:

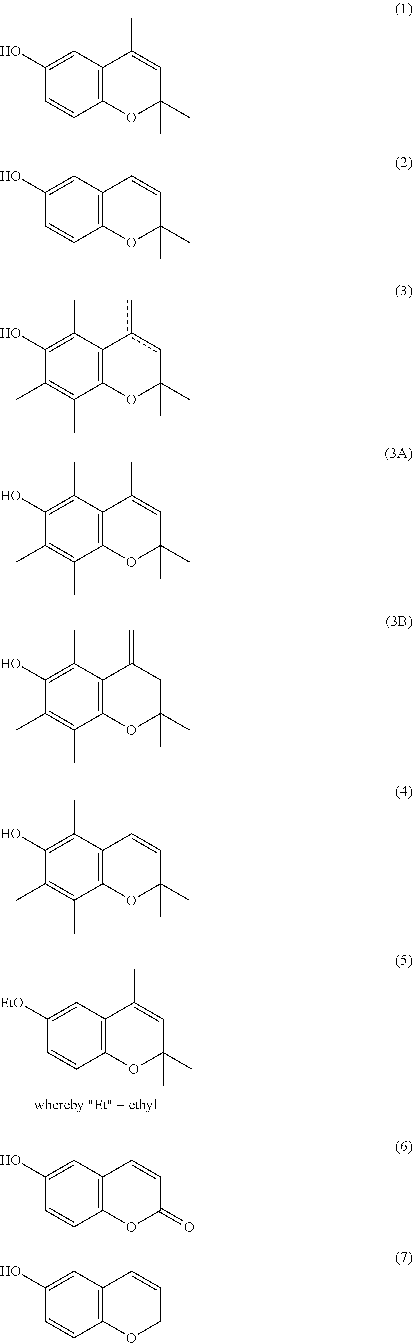

The compounds of the present invention are efficient as antioxidants, preferably in feed and feed ingredients.

Non-limiting examples of feed are pet food, feed for aquatic animals, feed for terrestrial animals such as poultry and pigs, and feed for insects.

Non-limiting examples of feed ingredients are poultry meal, fish meal, insect meal and PUFA-containing oil.

“PUFA(s)” means polyunsaturated fatty acid(s) such as docosahexaenoic acid (“DHA”) and/or eicosapentaenoic acid (“EPA”) and/or docosapentaenoic acid (“DPA”) and/or oleic acid and/or stearidonic acid and/or linoleic acid and/or alpha-linolenic acid (“ALA”) and/or gamma-linolenic acid and/or arachidonic acid (“ARA”) and/or the esters of all of them, whereby the term “esters” encompasses monoglycerides, diglycerides and triglycerides as well as C₁₋₆-alkyl esters such as especially the methyl esters and the ethyl esters, whereby the triglycerides are often dominant.

DHA, EPA, ALA and stearidonic acid are omega-3 fatty acids, whereas linoleic acid, gamma-linolenic acid and ARA are omega-6 fatty acids.

The term “DPA” encompasses two isomers, the omega-3 fatty acid clupanodonic acid (7Z,10Z,13Z,16Z,19Z-docosapentaenoic acid) and the omega-6 fatty acid osbond acid (4Z,7Z,10Z,13Z,16Z-docosapentaenoic acid).

In accordance with the invention, the polyunsaturated fatty acid (PUFA) is preferably DHA and/or EPA and/or DPA and/or any ester thereof, more preferably the polyunsaturated fatty acid (PUFA) is preferably DHA and/or EPA and/or any ester thereof.

Examples of PUFA-containing oils are
marine oil, such as preferably fish oil,
microbial biomass containing polyunsaturated fatty acids and/or their esters (“microbial oil”), preferably containing high amounts of docosahexaenoic acid (“DHA”) and/or eicosapentaenoic acid (“EPA”) and/or docosapentaenoic acid (“DPA”) and/or their esters, and
oil containing high amounts of PUFAs and/or their esters, preferably containing high amounts of docosahexaenoic acid (“DHA”) and/or eicosapentaenoic acid (“EPA”) and/or docosapentaenoic acid (“DPA”) and/or their esters, extracted from microbial biomass, such as fungae (“fungal oil”) or algae (“algal oil”), and
plant oil with relatively high amounts of PUFAs and/or their esters, (“PUFA-containing plant oil”), such as e.g. canola seed oil, linseed/flaxseed oil, hempseed oil, pumpkin seed oil, evening primrose oil, borage seed oil, blackcurrent seed oil, sallow thorn/sea buckthorn oil, chia seed oil, argan oil and walnut oil.

Thus, in addition, the present invention is

(1) directed to the use of a compound of formula (I) and/or a compound of formula (II) as antioxidant(s) in feed, such as especially feed for aquatic animals, feed for terrestrial animals such as poultry, pigs and pets, and feed for insects; as well as

(2) directed to the use of a compound of formula (I) and/or a compound of formula (II) as antioxidant(s) in feed ingredients, such as especially poultry meal, fish meal, insect meal and PUFA-containing oil, and

(3) directed to feed, such as especially feed for aquatic animals, feed for terrestrial animals such as poultry, pigs and pets, and feed for insects, comprising a compound of formula (I) and/or a compound of formula (II), and

(4) directed to feed ingredients, such as especially poultry meal, fish meal, insect meal and PUFA-containing oil, comprising a compound of formula (I) and/or a compound of formula (II).

Thus, the present invention is directed to feed for aquatic animals comprising a compound of formula (I) and/or a compound of formula (II) with the preferences as given above.

The present invention is also directed to feed for insects and terrestrial animals, e.g. pigs, poultry and pets, comprising a compound of formula (I) and/or a compound of formula (II) with the preferences as given above.

Aquatic animals in the context of the present invention encompass farmed crustacea such as shrimp and carnivorous species of farmed fish such as salmons, rainbow trout, brown trout (Salmo trutta) and gilthead seabream.

Thus, the feed for aquatic animals comprising at least one compound of formula (I) and/or at least one compound of formula (II) are especially fed to the aquatic animals as cited above.

I. Feed Ingredients

Feed ingredients are broadly classified into cereal grains, protein meals, fats and oils, minerals, feed additives, and miscellaneous raw materials, such as roots and tubers.

Further Antioxidants

The compounds of formulae (I) and/or (II) can be used in combination with one or more other antioxidants as described below.

In an embodiment of the present invention the feed ingredients of the present invention additionally comprise a mixture of 2-tert-butyl-4-methoxyphenol and 3-tert-butyl-4-methoxyphenol, which is known under the name “BHA” (butylated hydroxyanisole).

In a further embodiment of the present invention the feed ingredients of the present invention additionally comprise ascorbyl palmitate.

In another embodiment of the present invention the feed ingredients of the present invention additionally comprise BHA and ascorbyl palmitate.

Instead of ascorbyl palmitate other esters of ascorbic acid such as the esters of ascorbic acid with linear C₁₂₋₂₀ alkanols, preferably the esters of ascorbic acid with linear C₁₄₋₁₈ alkanols, may also be used, so that further embodiments of the present invention are directed to feed ingredients that additionally comprise esters of ascorbic acid with linear C₁₂₋₂₀ alkanols, preferably esters of ascorbic acid with linear C₁₄₋₁₈ alkanols, more preferably ascorbyl palmitate, whereby optionally BHA may also be present.

The feed ingredients may also comprise additionally alpha-tocopherol and/or gamma-tocopherol, whereby either an ester of ascorbic acid with a linear C₁₂₋₂₀ alkanol with the preferences as given above or BHA or both may additionally be present.

The feed ingredients themselves are described in more detail below.

1. PUFA-Containing Oils

In the context of the present invention the term “PUFA-containing oil” encompasses
marine oil, such as especially fish oil,
microbial biomass containing polyunsaturated fatty acids (“PUFAs”), especially docosahexaenoic acid (“DHA”) and/or eicosapentaenoic acid (“EPA”) and/or docosapentaenoic acid (“DPA”) and/or their esters (“microbial oil”);
oil containing high amounts of PUFAs, especially containing high amounts of DHA and/or EPA and/or DPA and/or their esters extracted from microbial biomass as e.g., fungi (“fungal oil”) or algae (“algal oil”);
Plant oil with high amounts of PUFAs and/or their esters (“PUFA-containing plant oil”), such as e.g. canola seed oil, linseed/flaxseed oil, hempseed oil, pumpkin seed oil, evening primrose oil, borage seed oil, blackcurrent seed oil, sallow thorn/sea buckthorn oil, chia seed oil, argan oil and walnut oil.

The term “DHA” does not only encompass the acid but also derivatives thereof such as monoglycerides, diglycerides and triglycerides as well as C₁₋₆-alkyl esters such as the methyl and ethyl esters. The same applies for “EPA” and “DPA” and all the other PUFAs.

Fish oil and algal oil are common feed ingredients. Instead of fish oil and algal oil also the other PUFA-containing oils named above may be used as feed ingredients, i.e.:
microbial biomass containing PUFAs (“microbial oil”)
oil containing high amounts of PUFAs extracted from microbial biomass, such as especially fungal oil, and
plant oil with high amounts of PUFAs.

The above-mentioned feed ingredients may not only be used as alternative of fish oil and algal oil, but also in addition.

Examples of PUFA-containing oils that are used as feed ingredients are given below in more detail.

Marine Oil

Examples of suitable marine oils include, but are not limited to, Atlantic fish oil, Pacific fish oil, or Mediterranean fish oil, or any mixture or combination thereof.

In more specific examples, a suitable fish oil can be, but is not limited to, pollack oil, bonito oil, pilchard oil, tilapia oil, tuna oil, sea bass oil, halibut oil, spearfish oil, barracuda oil, cod oil, menhaden oil, sardine oil, anchovy oil, capelin oil, herring oil, mackerel oil, salmonid oil, tuna oil, and shark oil, including any mixture or combination thereof.

Other marine oils suitable for use herein include, but are not limited to, squid oil, cuttle fish oil, octopus oil, krill oil, seal oil, whale oil, and the like, including any mixture or combination thereof.

For stabilizing marine oil an amount of at least one compound of formula (I) and/or at least one compound of formula (II) ranging from 10 to 500 ppm, preferably ranging from 30 to 300 ppm, more preferably ranging from 100 to 250 ppm, based on the total amount of the marine oil, is usually sufficient. The same applies for the other PUFA-containing oils such as microbial oil, algal oil, fungal oil and PUFA-containing plant oil.

A commercially available example of marine oil is the fish oil “MEG-3” (Bleached 30S TG Fish oil) from DSM Nutritional Products, LLC (US) whose specification and composition is shown in Tables I and II below:

TABLE 1

| ANALYSIS | SPECIFICATIONS |
|---|---|
| Colour | Max. 6 Gardner Colour |
| Free Fatty Acid (as % Oleic) | Max. 0.4% |
| p-Anisidine Value | Max. 12 (at time of release) |
| Peroxide Value | Max. 3 milli equivalents/kg |
| | (at time of release) |
| % Moisture | Max. 0.05% |
| Cold Test | Remains clear at 0° C. for 3 hours |
| Cholesterol | Report Actual |
| TOTOX ((2 × Peroxide Value) + | Max. 20 |
| (p-Anisidine Value)) | |

The peroxide value is defined as the amount of peroxide oxygen per 1 kilogram of oil. Traditionally this is expressed in units of milliequivalents or meq/kg.

Winterization is part of the processing of fish oil, and it is performed to remove solid fat in the oil. The “cold test” is performed to check if any solid fat is present and precipitated in the oil when cooled to 0° C. within a specific period of time. In this fish oil (Product Code: FG30TG), any such precipitation is checked for 3 hours at 0° C.

TABLE II

| Fatty Acid Profile | |
|---|---|
| EPA (A %) | Min. 18 |
| EPA mg/g (as TG) | Min. 170 |
| DHA (A %) | Min. 12 |
| DHA mg/g (as TG) | Min. 110 |
| EPA + DHA (A %) | Min. 30 |
| Total Omega 3 (A %) | Min. 34 |

“TG”=triglyceride;

“A %”=“area %”= area percentage by GC based on 24 peak analysis (meaning the 24 highest peaks have been analyzed)

Oil Containing High Amounts of PUFAs, Especially Containing High Amounts of DHA and/or EPA and/or DPA and/or their Esters, Extracted from Microbial Biomass as e.g., Fungi (“Fungal Oil”) or Algae (“Algal Oil”)

Algal Oil

“Algal oil” is an oil containing high amounts of DHA and/or EPA and/or DPA and/or their esters extracted from algae as microbial source/biomass.

An example of algal oil is the commercially available “Algal oil containing EPA+DPA” from DSM Nutritional Products, LLC (US) whose composition is shown in the Table III below:

TABLE III

| Fatty Acid Profile | |
|---|---|
| DHA + EPA content, mg/g oil | 587 mg/g |
| DHA content, mg/g oil | 401 mg/g |
| EPA content, mg/g oil | 186 mg/g |
| TOTOX ((2 × Peroxide Value) + | 5 |
| (p-Anisidine Value)) | |
| Free Fatty Acid | 0.6% |
| Moisture | <0.05% |

A further example of a crude oil containing high amounts of DHA and/or EPA extracted from microbial sources as e.g., algae, is the oil extracted from Algae Schizochytrium Biomass, whose specification is given in the following Table IV.

TABLE IV

| Specification | Aqua (Base Product) |
|---|---|
| DHA + EPA, mg/g oil | minimal 500 mg/g |
| DHA content, mg/g oil | minimal 250 mg/g |
| | (at least 25%->40%) |
| EPA content, mg/g oil | minimal 100 mg/g |
| | (at least 10%->25%) |
| Minimal ratio EPA:DHA | 1:4 |
| Maximal ratio EPA:DHA | 1:1 |
| TOTOX ((2 × Peroxide Value) + | maximum 35 |
| (p-Anisidine Value)) | |
| Free fatty acid | maximal 5% |
| Moisture | maximal 0.75% |
| DPA n-3 (omega-3 | <6 |
| docosapentaenoic acid), % | |
| Arachidonic Acid, % | <2 |
| Stearic, % | <2.5 |
| Palmitic, % | <30 |
| Shelf life | 6 months at 25° C. |
| Total Fat | Record |
| Crude Fat | >92% |

Microbial Biomass Containing Polyunsaturated Fatty Acids (“PUFAs”), Especially Docosahexaenoic Acid and/or Eicosapentaenoic Acid and/or Docosapentaenoic Acid (“DPA”) and/or their Esters

The biomass preferably comprises cells which produce PUFAs hetero-trophically. According to the invention, the cells are preferably selected from algae, fungi, particularly yeasts, bacteria, or protists. The cells are more preferably microbial algae or fungi.

Suitable cells of oil-producing yeasts are, in particular, strains of Yarrowia, Candida, Rhodotorula, Rhodosporidium, Cryptococcus, Trichosporon and Lipomyces.

Oil produced by a microorganism or obtained from a microbial cell is referred to as “microbial oil”. Oil produced by algae and/or fungi is referred to as an algal and/or a fungal oil, respectively.

As used herein, a “microorganism” refers to organisms such as algae, bacteria, fungi, protist, yeast, and combinations thereof, e.g., unicellular organisms. A microorganism includes but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; yeast (Ascomycetes or Basidiomycetes); and fungi of the genera Mucor, Mortierella, including but not limited to Mortierella alpina and Mortierella sect. schmuckeri, and Pythium, including but not limited to Pythium insidiosum.

In one embodiment, the microorganisms of the kingdom Stramenopiles may in particular be selected from the following groups of microorganisms: Hamatores, Proteromonads, Opalines, Developayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinales, Pedinellales, Dictyochales), Chrysomeridales, Sarcinochrysidales, Hydrurales, Hibberdiales, and Chromulinales.

In one embodiment, the microorganisms are from the genus Mortierella, genus Crypthecodinium, genus Thraustochytrium, and mixtures thereof. In a further embodiment, the microorganisms are from Crypthecodinium Cohnii. In a further embodiment, the microorganisms are from Mortierella alpina. In a still further embodiment, the microorganisms are from Schizochytrium sp. In yet an even further embodiment, the microorganisms are selected from Crypthecodinium Cohnii, Mortierella alpina, Schizochytrium sp., and mixtures thereof.

In a still further embodiment, the microorganisms include, but are not limited to, microorganisms belonging to the genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium, and genus Saprolegnia.

In an even further embodiment, the microorganisms are from microalgae of the order Thraustochytriales, which includes, but is not limited to, the genera Thraustochytrium (species include arudimentale, aureum, benthicola, globosum, kinnei, motivum, multirudimentale, pachydermum, proliferum, roseum, striatum); the genera Schizochytrium (species include aggregatum, limnaceum, mangrovei, minutum, octosporum); the genera Ulkenia (species include amoeboidea, kerguelensis, minuta, profunda, radiate, sailens, sarkariana, schizochytrops, visurgensis, yorkensis); the genera Aurantiacochytrium; the genera Oblongichytrium; the genera Sicyoidochytium; the genera Parientichytrium; the genera Botryochytrium; and combinations thereof. Species described within Ulkenia will be considered to be members of the genus Schizochytrium. In another embodiment, the microorganisms are from the order Thraustochytriales. In yet another embodiment, the microorganisms are from Thraustochytrium.

In still a further embodiment, the microorganisms are from Schizochytrium sp.

In certain embodiments, the oil can comprise a marine oil. Examples of suitable marine oils are the ones as given above.

The biomass according to the invention preferably comprises cells, and preferably consists essentially of such cells, of the taxon Labyrinthulomycetes (Labyrinthulea, net slime fungi, slime nets), in particular, those from the family of Thraustochytriaceae. The family of the Thraustochytriaceae (Thraustochytrids) includes the genera Althomia, Aplanochytrium, Aurantiochytrium, Botryochytrium, Elnia, Japonochytrium, Oblongichytrium, Parietichytrium, Schizochytrium, Sicyoidochytrium, Thraustochytrium, and Ulkenia. The biomass particularly preferably comprises cells from the genera Aurantiochytrium, Oblongichytrium, Schizochytrium, or Thraustochytrium, more preferably from the genus Schizochytrium.

In accordance with the invention, the polyunsaturated fatty acid (PUFA) is preferably DHA and/or EPA and/or their esters as defined above.

The cells present in the biomass are preferably distinguished by the fact that they contain at least 20 weight-%, preferably at least 30 weight-%, in particular at least 35 weight-%, of PUFAs, in each case based on cell dry matter.

In a very preferred embodiment of the current invention, cells, in particular a Schizochytrium strain, is employed which produces a significant amount of EPA and DHA, simultaneously, wherein DHA is preferably produced in an amount of at least 20 weight-%, preferably in an amount of at least 30 weight-%, in particular in an amount of 30 to 50 weight-%, and EPA is produced in an amount of at least 5 weight-%, preferably in an amount of at least 10 weight-%, in particular in an amount of 10 to 20 weight-% (in relation to the total amount of lipid as contained in the cells, respectively).

Preferred species of microorganisms of the genus Schizochytrium, which produce EPA and DHA simultaneously in significant amounts, as mentioned before, are deposited under ATCC Accession No. PTA-10208, PTA-10209, PTA-10210, or PTA-10211, PTA-10212, PTA-10213, PTA-10214, PTA-10215.

DHA and EPA producing Schizochytrium strains can be obtained by consecutive mutagenesis followed by suitable selection of mutant strains which demonstrate superior EPA and DHA production and a specific EPA:DHA ratio. Any chemical or nonchemical (e.g. ultraviolet (UV) radiation) agent capable of inducing genetic change to the yeast cell can be used as the mutagen. These agents can be used alone or in combination with one another, and the chemical agents can be used neat or with a solvent.

Methods for producing the biomass, in particular, a biomass which comprises cells containing lipids, in particular PUFAs, particularly of the order Thraustochytriales, are described in detail in the prior art (see e.g. WO 91/07498, WO 94/08467, WO 97/37032, WO 97/36996, WO 01/54510). As a rule, the production takes place by cells being cultured in a fermenter in the presence of a carbon source and a nitrogen source, along with a number of additional substances like minerals that allow growth of the microorganisms and production of the PUFAs. In this context, biomass densities of more than 100 grams per litre and production rates of more than 0.5 gram of lipid per litre per hour may be attained. The process is preferably carried out in what is known as a fed-batch process, i.e. the carbon and nitrogen sources are fed in incrementally during the fermentation. When the desired biomass has been obtained, lipid production may be induced by various measures, for example by limiting the nitrogen source, the carbon source or the oxygen content or combinations of these.

In a preferred embodiment of the current invention, the cells are grown until they reach a biomass density of at least 80 or 100 g/l, more preferably at least 120 or 140 g/l, in particular at least 160 or 180 g/l (calculated as dry-matter content). Such processes are for example disclosed in U.S. Pat. No. 7,732,170.

Preferably, the cells are fermented in a medium with low salinity, in particular, so as to avoid corrosion. This can be achieved by using chlorine-free sodium salts as the sodium source instead of sodium chloride, such as, for example, sodium sulphate, sodium carbonate, sodium hydrogen carbonate or soda ash. Preferably, chloride is used in the fermentation in amounts of less than 3 g/l, in particular, less than 500 mg/l, especially preferably less than 100 mg/l.

PUFA-Containing Plant Oils: Plant Oils with Relatively High Amounts of PUFAs, Especially with High Amounts of DHA and/or EPA Such as e.g., Canola Seed Oil

The plant cells may, in particular, be selected from cells of the families Brassicaceae, Elaeagnaceae and Fabaceae. The cells of the family Brassicaceae may be selected from the genus Brassica, in particular, from oilseed rape, turnip rape and Indian mustard; the cells of the family Elaeagnaceae may be selected from the genus Elaeagnus, in particular, from the species Oleae europaea; the cells of the family Fabaceae may be selected from the genus Glycine, in particular, from the species Glycine max.

### Examples

Canola seed oil with a content of DHA of at least 9% by weight, of at least 12% by weight, of at least 15% by weight, or of at least 20% by weight, based on the total weight of the canola seed oil;
Canola seed oil with a content of EPA of at least 9% by weight, of at least 12% by weight, of at least 15% by weight, or of at least 20% by weight, based on the total weight of the canola seed oil.

Examples of PUFA-containing plant oils containing high amounts of other PUFAs than EPA and/or DHA and/or DPA and/or their esters are linseed/flaxseed oil, hempseed oil, pumpkin seed oil, evening primrose oil, borage seed oil, blackcurrent seed oil, sallow thorn/sea buckthorn oil, chia seed oil, argan oil and walnut oil.

2. Other Feed Ingredients

Poultry Meal/Chicken Meal

Poultry meal is a high-protein commodity used as a feed ingredient. It is made from grinding clean, rendered parts of poultry carcasses and can contain bones, offal, undeveloped eggs, and some feathers. Poultry meal quality and composition can change from one batch to another.

Chicken meal, like poultry meal, is made of “dry, ground, rendered clean parts of the chicken carcass” according to AAFCO and may contain the same ingredients as poultry meal. Chicken meal can vary in quality from batch to batch. Chicken meal costs less than chicken muscle meat and lacks the digestibility of chicken muscle meat.

Poultry meal contains preferably not less than 50 weight-% of crude protein, not less than 5 weight-% of crude fat, not more than 5 weight-% of crude fiber, not more than 40 weight-% of ash and not more than 15 weight-% of water, each based on the total weight of the poultry meal, whereby the total amount of all ingredients sums up to 100 weight-%.

More preferably poultry meal contains from 50 to 85 weight-% of crude protein, and from 5 to 20 weight-% of crude fat, and from 1 to 5 weight-% of crude fiber, and from 5 to 40 weight-% of ash, and from 5 to 15 weight-% of water, each based on the total weight of the poultry meal, whereby the total amount of all ingredients sums up to 100 weight-%.

For stabilizing poultry meal an amount of at least one compound of formula (I) and/or at least one compound of formula (II) ranging from 10 to 1000 ppm, preferably ranging from 30 to 700 ppm, more preferably ranging from 100 to 500 ppm, based on the total amount of the poultry meal, is usually sufficient.

The same amounts also apply for chicken meal.

Fish Meal

Fish meal contains preferably not less than 50 weight-% of crude protein, and not more than 20 weight-% of crude fat, and not more than 10 weight-% of crude fibers, and not more than 25 weight-% of ash, and not more than 15 weight-% of water, each based on the total weight of the fish meal, whereby the total amount of all ingredients sums up to 100 weight-%.

More preferably fish meal contains from 50 to 90 weight-% of crude protein and from 5 to 20 weight-% of crude fat, and from 1 to 10 weight-% of crude fibers, and from 5 to 25 weight-% of ash, and from 5 to 15 weight-% of water, each based on the total weight of the fish meal, whereby the total amount of all ingredients sums up to 100 weight-%.

For stabilizing fish meal an amount of at least one compound of formula (I) and/or at least one compound of formula (II) ranging from 10 to 2000 ppm, preferably ranging from 100 to 1500 ppm, more preferably ranging from 300 to 1000 ppm, based on the total amount of the fish meal, is usually sufficient.

Fish meal is a commercial product made from fish that is used primarily as a protein supplement in compound feed, especially for feeding farmed fish, crustacea, pigs and poultry, and companion animals such as cats and dogs.

A portion of the fish meal is made from the bones and offal left over from processing fish used for human consumption, while the larger percentage is manufactured from wild-caught, small marine fish. It is powder or cake obtained by drying the fish or fish trimmings, often after cooking, and then grinding it. If the fish used is a fatty fish it is first pressed to extract most of the fish oil.

The uses and need of fish meal are increasing due to the rising demand for fish, because fish has the best feed conversion rate of all farmed animals, can be produced well in developing countries and has a small size, i.e. can be slaughtered for preparing a meal, so that there is no need to store the fish. Furthermore, there are no religious constraints concerning the consumption of fish, fish is a source of high quality protein and it is easy to digest.

Fish meal is made by cooking, pressing, drying, and grinding of fish or fish waste to which no other matter has been added. It is a solid product from which most of the water is removed and some or all of the oil is removed. About four or five tons of fish are needed to manufacture one ton of dry fish meal.

Of the several ways of making fish meal from raw fish, the simplest is to let the fish dry out in the sun. This method is still used in some parts of the world where processing plants are not available, but the end-product is of poor quality in comparison with ones made by modern methods.

Currently, all industrial fish meal is usually made by the following process:

Cooking: A commercial cooker is a long, steam-jacketed cylinder through which the fish are moved by a screw conveyor. This is a critical stage in preparing the fishmeal, as incomplete cooking means the liquid from the fish cannot be pressed out satisfactorily and overcooking makes the material too soft for pressing. No drying occurs in the cooking stage.

Pressing: A perforated tube with increasing pressure is used for this process. This stage involves removing some of the oil and water from the material and the solid is known as press cake. The water content in pressing is reduced from 70% to about 50% and oil is reduced to 4%.

Drying: If the fish meal is under-dried, moulds or bacteria may grow. If it is over-dried, scorching may occur and this reduces the nutritional value of the meal.

The two main types of dryers are:

Direct: Very hot air at a temperature of about 500° C. is passed over the material as it is tumbled rapidly in a cylindrical drum. This is the quicker method, but heat damage is much more likely if the process is not carefully controlled.

Indirect: A cylinder containing steam-heated discs is used, which also tumbles the meal.

Grinding: This last step in processing involves the breakdown of any lumps or particles of bone.

The fish meal has to be transported long distances by ship or other vehicles to the various locations, where it is used.

Unmodified fish meal can spontaneously combust from heat generated by oxidation of the polyunsaturated fatty acids in the fish meal. Therefore, it has to be stabilized by antioxidants. Especially advantageous for this purpose are the compounds of formulae (I) and/or (II) of the present invention.

Insect Meal

Insect meal has a high content of protein and is therefore, a valuable source of protein.

In general any insect may be manufactured to meal, but insects of special interest in the context of the present invention encompass black soldier flies (Hermetia species, commonly called BSF), mealworms (Tenebrio molitor), lesser mealworms (Alphitobius diaperinus), house cricket (Acheta domesticus, grasshoppers (Locusta migratoria), buffaloworms (Alphitobius diaperinus), cockroaches and domestic flies, whereby black soldier flies (Hermetia species, commonly called BSF), mealworms (Tenebrio molitor) and lesser mealworms (Alphitobius diaperinus) are more preferred.

For stabilizing insect meal an amount of at least one compound of formula (I) and/or at least one compound of formula (II) ranging from 10 to 1000 ppm, preferably ranging from 30 to 700 ppm, more preferably ranging from 100 to 500 ppm, based on the total amount of the insect meal, is usually sufficient.

II. Feed

The compounds of formula (I) and/or the compounds of formula (II) are not only suitable for stabilizing feed ingredients such as poultry meal, fish meal, insect meal and PUFA-containing oil, but also effective antioxidants for feed.

Feed (or ‘feedingstuff’) means any substance or product, including additives, whether processed, partially processed or unprocessed, intended to be used for oral feeding to animals.

Feed in the context of the present invention is feed for aquatic animals and for terrestrial animals, as well as feed for insects.

For stabilizing feed an amount of at least one compound of formula (I) and/or at least one compound of formula (II) ranging from 10 to 500 ppm, preferably ranging from 30 to 300 ppm, more preferably ranging from 100 to 250 ppm, based on the total amount of the feed, is usually sufficient.

Further Antioxidants

The compounds of formulae (I) and/or (II) can be used in combination with one or more other antioxidants as described below.

In an embodiment of the present invention the feed of the present invention additionally comprises a mixture of 2-tert-butyl-4-methoxyphenol and 3-tert-butyl-4-methoxyphenol, which is known under the name “BHA” (butylated hydroxyanisole).

In a further embodiment of the present invention the feed of the present invention additionally comprises ascorbyl palmitate.

In another embodiment of the present invention the feed of the present invention additionally comprises BHA and ascorbyl palmitate.

Instead of ascorbyl palmitate other esters of ascorbic acid such as the esters of ascorbic acid with linear C₁₂₋₂₀ alkanols, preferably the esters of ascorbic acid with linear C₁₄₋₁₈ alkanols, may also be used, so that further embodiments of the present invention are directed to feed that additionally comprises esters of ascorbic acid with linear C₁₂₋₂₀ alkanols, preferably esters of ascorbic acid with linear C₁₄₋₁₈ alkanols, more preferably ascorbyl palmitate, whereby optionally BHA may also be present.

The feed may also comprise additionally alpha-tocopherol and/or gamma-tocopherol, whereby either an ester of ascorbic acid with a linear C₁₂₋₂₀ alkanol with the preferences as given above or BHA or both may additionally be present.

The feed itself is described in more detail below.

Feed for Poultry

The feed for poultry differs from region to region. In the following Tables V and VI typical examples for diets in Europe and Latin America are given. These diets include cereals such as wheat, rye, maize/corn, minerals such as NaCl, vegetable oils such as soya oil, amino acids and proteins.

TABLE V
European diet

| | Starter Period | Grower Period |
|---|---|---|
| Ingredients (%) | (day 0-21) | (day 22-36) |
| Wheat | 20.00 | 22.50 |
| Rye | 12.00 | 12.00 |
| Soybean meal | 34.00 | 28.50 |
| Maize | 27.00 | 28.50 |
| Vegetable Oil | 3.10 | 4.20 |
| NaCl | 0.10 | 0.10 |
| DL Methionine | 0.24 | 0.24 |
| L-Lysine | 0.15 | 0.15 |
| Limestone | 0.85 | 0.85 |
| Dicalcium Phosphate | 1.50 | 1.90 |
| Vitamin & Mineral mix | 1.00 | 1.00 |
| Coccidiostat (Avatec) | 0.06 | 0.06 |
| TiO₂ | — | 0.10 |
| calculated Provision | | |
| apparent metabolizable | 12.5 | 12.90 |
| energy, MJ/kg | | |
| apparent metabolizable | 2986 | 3082 |
| energy, kcal/kg | | |
| crude Protein, % | 21.2 | 19.1 |
| Methionine + Cysteine, % | 0.89 | 0.83 |
| Lysine, % | 1.23 | 1.09 |
| Calcium, % | 0.83 | 0.91 |
| total phosphorus, % | 0.68 | 0.73 |
| available phosphorus, % | 0.35 | 0.40 |

TABLE VI
Latin American diet

| Ingredients (%) | Starter | Grower |
|---|---|---|
| Corn | 53.0 | 57.1 |
| Soybean meal | 38.5 | 34.2 |
| Calcium | 0.70 | 0.70 |
| Phosphorus | 2.40 | 2.00 |
| NaHCO₃ | 0.23 | 0.24 |
| NaCl | 0.20 | 0.20 |
| Methionine | 0.30 | 0.10 |
| Lysine | 0.21 | 0.00 |
| Soya Oil | 3.50 | 4.50 |
| Premix | 1.00 | 1.00 |
| Calculated provision (%) | | |
| Crude protein | 22.4 | 20.4 |
| apparent metabolizable energy, (MJ/kg) | 12.7 | 13.2 |
| apparent metabolizable energy, (kcal/kg) | 3034 | 3154 |
| Total phosphorus | 0.86 | 0.76 |
| Calcium | 1.00 | 0.85 |
| Available phosphorus | 0.44 | 0.38 |
| d-Lysine | 1.25 | 0.98 |
| d-Methionine + Cysteine | 0.91 | 0.68 |
| d-Threonine | 0.77 | 0.71 |
| Na | 0.18 | 0.18 |
| Cl | 0.20 | 0.19 |

Pet Food

Pet foods are formulated to meet nutrient specifications using combinations of multiple ingredients to meet the targeted nutrient specification.

Poultry meal e.g. is an ingredient that is commonly found in Dog and Cat foods.

The nutrient specifications for a complete and balanced dog or cat food will meet or exceed the guidelines provided by AAFCO (American Association of Feed Control Officials). The ingredient composition of pet-food can include any legal feed ingredient so number of combinations are not quite infinite but close. Some examples of ingredient used in dog and cat foods can be found in Table VII below:

TABLE VII

| Ingredient Class/Ingredient | Use rates |
|---|---|
| 1 ANIMAL MEALS | 10-35% |
| Chicken | |
| Turkey | |
| Duck | |
| Poultry Br-Product | |
| Lamb | |
| Venison | |
| Beef | |
| Pork | |
| Meat & Bone | |
| Fish | |
| 2 FRESH MEATS | 3-20% |
| Chicken | |
| Turkey | |
| Duck | |
| Lamb | |
| Venison | |
| Beef | |
| Pork | |
| Fish | |
| 3 VEGETABLE PROTEINS | 8-20% |
| Soybean Meal | |
| Corn Gluten Meal | |
| Pea Protein | |
| Potato Protein | |
| Soy Protein Conc/Isolates | |
| 4 GRAINS | 0-70% |
| Corn/Maize | |
| Wheat | |
| Brown Rice/Brewers Rice | |
| Oatmeal/Oat Groats | |
| Barley | |
| Millet | |
| Milo/Sorghum | |
| Rye | |
| Corn Gluten Feed | |
| Wheat Middlings | |
| 5 FIBER SOURCES | 2-8% |
| Beet Pulp | |
| Corn Bran | |
| Wheat Bran | |
| Cellulose | |
| Tomato Ponace | |
| Potato Fiber | |
| Pea Fiber | |
| 6 FATS & OILS | 1-15% |
| Animal Fat | |
| Poultry Fat | |
| Chicken Fat | |
| Beef Tallow | |
| Sunflower Oil | |
| Canola Oil | |
| 7 MICRONUTRIENTS | 0.10-1% |
| Vitamins | |
| Minerals | |
| Others (e.g. Fructooligosaccharides | |
| (FOS) used as a pre-biotic) | |
| 8 PALATANTS (FLAVORS) | 0-5% |
| 9 Other non-basic ingredients | |
| Dried Egg Product | 1-15% |
| Fish Oil | 0.5-2% |
| Fish Meal | 1-4% |
| Flaxseed | 1-4% |
| Dried Peas | 5-30% |
| Dried Chickpeas | 5-30% |
| Dried Lentils | 5-10% |
| Dried Potatoes | 5-20% |
| Dried Sweet Potatoes | 5-20% |
| Tapioca Starch | 5-15% |
| Potato Starch | 5-15% |
| Pea Starch | 5-15% |

For stabilizing pet food an amount of at least one compound of formula (I) and/or at least one compound of formula (II) ranging from 10 to 500 ppm, preferably ranging from 30 to 300 ppm, more preferably ranging from 100 to 250 ppm, based on the total amount of the pet food, is usually sufficient.

Feed for Fish

A typical example of feed for fish comprises the following ingredients, whereby all amounts are given in weight-%, based on the total weight of the feed for fish:
Fish meal in an amount ranging from 5 to 15 weight-%, preferably fish meal in said amount comprising at least one compound of formula (I) and/or at least one compound of formula (II) of the present invention as antioxidants;
fish hydrolysates in an amount ranging from 0 to 5 weight-%;
vegetable proteins in an amount ranging from 30 to 45 weight-%;
binders, mainly starch, in an amount ranging from 9 to 12 weight-%;
micro-ingredients such as vitamins, choline, minerals, mono calcium phosphate (“MCP”) and/or amino acids in an amount ranging from 3 to 6 weight-%;
marine oil in an amount ranging from 5 to 10 weight-%, preferably marine oil in said amount comprising at least one compound of formula (I) and/or at least one compound of formula (II) of the present invention as antioxidants;
vegetable oil in an amount ranging from 20 to 25 weight-%, preferably vegetable oil in said amount comprising at least one compound of formula (I) and/or at least one compound of formula (II) of the present invention as antioxidants;

and whereby the amount of all ingredients sum up to 100 weight-%.

For stabilizing feed for fish an amount of at least one compound of formula (I) and/or at least one compound of formula (II) ranging from 10 to 1000 ppm, preferably ranging from 30 to 700 ppm, more preferably ranging from 100 to 500 ppm, based on the total amount of the feed for fish, is usually sufficient.

Novel Compounds

The compounds of formulae (3) and (5) are novel. Therefore, the present invention is also directed to them and their synthesis. Compound of formula (3) is a mixture of compounds of formulae (3a) and (3b).

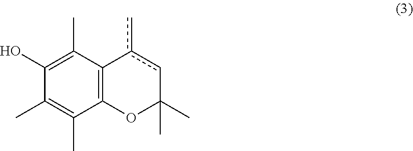

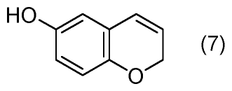

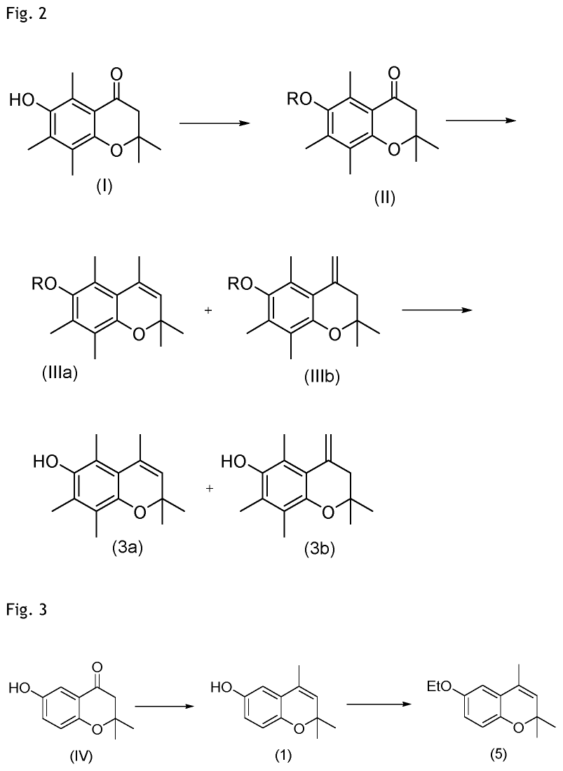

The synthesis of compound of formula (3) is shown in FIG. 2. Compound of formula (3) is a mixture of compounds of formulae (3a) and (3b), preferably in the molar ratio of 82:18.

The starting material for compound of formula (3), 6-hydroxy-2,2,5,7,8-pentamethylchroman-4-one, may be prepared according to US 2006/193797, Example 1.

The present invention is also directed to a process for the manufacture of a mixture of compounds of formulae (3a) and (3b) comprising the following steps:

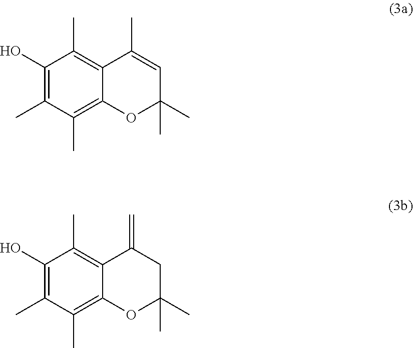 [SMILES: CC1=CC(C)(C)Oc2c(C)c(C)c(O)c(C)c21 ; C=C1CC(C)(C)Oc2c(C)c(C)c(O)c(C)c21]

a) optional protection of the hydroxy group of the compound of formula (I) to obtain the compound of formula (II),

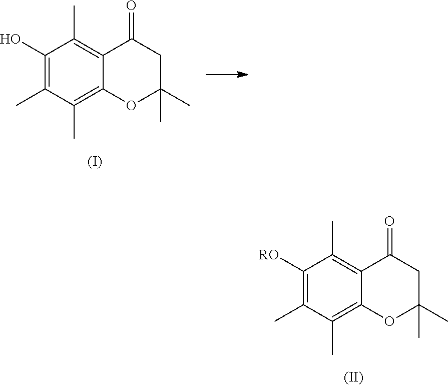

and

b) methyl-Grignard addition to the compound of formula (II) and water elimination to obtain a mixture of compounds of formulae (IIIa) and (IIIb); or methyl-Grignard addition to the compound of formula (I) and water elimination to obtain a mixture of compounds of formulae (3a) and (3b), respectively;

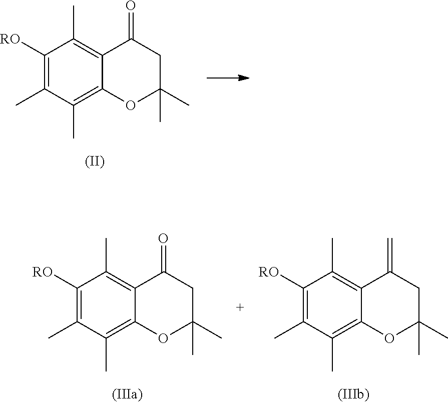

and

c) optional deprotection of the compounds of formulae (IIIa) and (IIIb) to a mixture of compounds of formulae (3a) and (3b)

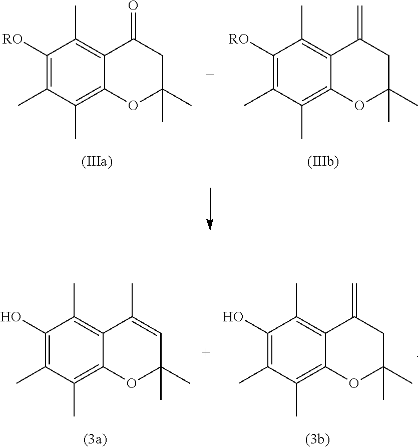

Step b):

The methyl-Grignard may be added to the compound of formula (II) (preferred) or to the compound of formula (I) (less preferred). In the latter case (methyl-Grignard addition to the compound of formula (I) double the amount of the methyl-Grignard reagent has to be used.

If the methyl-Grignard is added to the compound of formula (I) a mixture of compounds of formulae (IIIa) and (IIIb), wherein R═H is obtained in step b), i.e. a mixture of compounds of formula (3a) and (3b). In this case, step c) is not carried out.

Step a):

The hydroxy group of the compound of formula (I) is a phenolic group which may be protected as ether, acetal or ester, preferably as ether or acetal, according to state-of-the-art methods.

Examples of such phenol protecting groups are methyl ethers; benzyl ethers; silyl ethers, such as e.g. trimethylsilyl, tert-butyldimethylsilyl, triisopropylsilyl and triethylsilyl ethers; tetrahydropyran ethers (acetals); an acetal formed with isopropylene methyl ether (2-methoxy-2-propanyl acetal) and pivaloyl esters.

Step c):

The protected phenolic group (“OR” in the compound of formula (II)) may be deprotected easily, i.e. by state-of-the-art methods, to the phenolic group again.

The synthesis of compound of formula (5) is shown in FIG. 3. The starting material for compound of formula (5), 6-hydroxy-2,2-dimethylchroman-4-one (compound of formula (IV)), may be prepared according to C. L. Lucas, B. Lygo, A. J. Blake, W. Lewis, C. J. Moody. Regioselectivity of the Claisen Rearrangement in meta-Allyloxy Aryl Ketones: An Experimental and Computational Study, and Application in the Synthesis of (R)-(−)-Pestalotheol D. Chem. Eur. J. 2011, 17, 1972-1978.

A further embodiment of the present invention is a process for the manufacture of compound of formula (5) comprising the following steps:

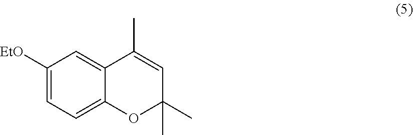 [SMILES: CCOc1ccc2c(c1)C(C)=CC(C)(C)O2]

whereby “Et” is ethyl,

i) methyl-Grignard addition to compound of formula (IV) and water elimination to obtain the compound of formula (1),

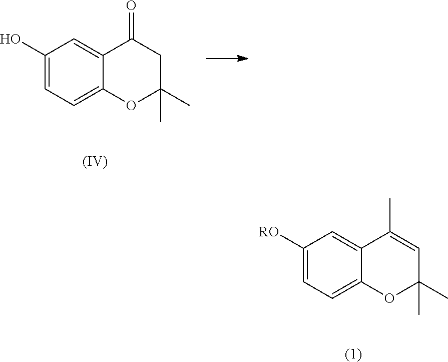

and

ii) etherification of compound of formula (1) to obtain the compound of formula (5)

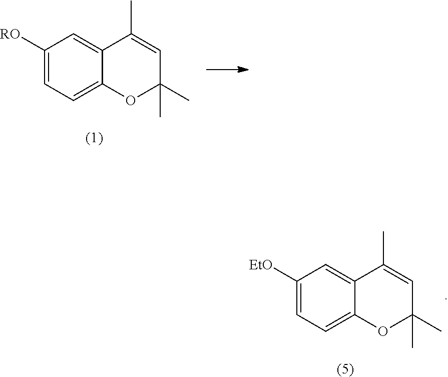

Step ii)

The etherification of compound of formula (1) to obtain the compound of formula (5) is either achieved by reaction with an ethyl halide (preferably chloride or bromide) or a carbonate such as diethyl carbonate or dimethyl carbonate.

### Preferred Embodiments of the Present Invention

The compounds of the present invention especially suitable for stabilizing fish meal and thus, preventing combusting of the fish meal and preserving its nutritional value, are compounds of formulae (1), (3), (4), (5), (6) and (7), whereby compounds of formulae (1), (3), (4), (5) and (6) are preferred, compounds of formulae (1), (3) and (4) are more preferred and compound of formula (1) is the most preferred.

The compounds especially suitable for stabilizing poultry meal are compounds of formulae (1) and (2).

The compound especially suitable for stabilizing pet food is compound of formula (1).

Fish Oil/Algal Oil

The Protection Factors of compound of formula (1) in fish oil could be improved by the addition of ascorbyl palmitate (“AP”) (see Table 10 in the experimental part) indicating the possibility of combining AP to all the compounds of formulae (1) to (7) to improve the oxidative stability of matrices containing high amounts of unsaturated fatty acids such as fish oil.

Polymers are combination of complex compounds generated at the end of the oxidation cascade of unsaturated fatty acids and, they indicate the levels of overall oxidation of the matrix. The generation of such polymers in fish oil containing these novel antioxidant compounds of formulae (1) to (7) could be reduced considerably when AP was added as a synergistically acting compound (see Table 11 in the experimental part).

The invention is now further illustrated in the following non-limiting examples.

### Examples

### Examples 1-6: Syntheses of Compounds of Formulae (1) to (5) and (7)

Compound of formula (6), 6-hydroxycoumarin, is commercially available, e.g. from Aldrich, catalog#642665.

The following abbreviations have been used:

min minute(s)

h hour(s)

DBU 1,8-diazabicyclo[5.4.0]undec-7-ene

DCM dichloromethane

DMF dimethylformamide

MeOH methanol

THF tetrahydrofuran

### Example 1: Synthesis of Compound of Formula (1) (2,2,4-trimethyl-2H-chromen-6-ol

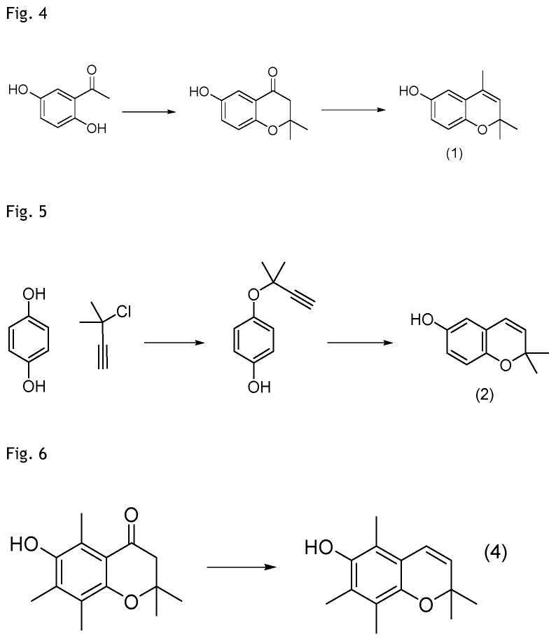

See FIG. 4:

Step 1: acetone, pyrrolidine, acetonitrile;

Step 2: 1) MeMgCl, THF, 2) HCl, pTsOH.

For step 1, a procedure by Moody was followed (C. L. Lucas, B. Lygo, A. J. Blake, W. Lewis, C. J. Moody. Regioselectivity of the Claisen Rearrangement in meta-Allyloxy Aryl Ketones: An Experimental and Computational Study, and Application in the Synthesis of (R)-(−)-Pestalotheol D. Chem. Eur. J. 2011, 17, 1972-1978).

Step 2: A 750 mL 4-necked sulfonation flask equipped with mechanical stirrer, thermometer, 250 mL addition funnel, argon inlet and reflux condenser was inertized with argon and then charged with MeMgCl (3.0 M solution in THF, 128 mL, 384 mmol, 2.4 mol equiv.) at 20° C. Subsequently, a solution of 6-hydroxy-2,2-dimethylchroman-4-one (33.9 g, 160 mmol) in dry THF (160 mL) was charged into the addition funnel. The solution was then added over 35 min, keeping the inner temperature below 20° C. The reaction was then heated to reflux for 3 h. Additional MeMgCl (3.0 M solution in THF, 32 mL, 96 mmol, 0.6 mol equiv.) was added. The reaction was heated to reflux for another 1.75 h, then cooled to room temperature and stirred overnight. Subsequently, the mixture was slowly quenched by addition of HCl (4 M in water, 132 mL, 528 mmol, 3.3 mol equiv.) over 30 min, keeping the inner temperature below 30° C. p-Toluenesulfonic acid monohydrate (0.30 g, 1.6 mmol, 1 mol %) was added and the mixture was heated to reflux for 1 h. The reaction was cooled to room temperature and diluted with EtOAc (150 mL). After phase separation, the aqueous phase was extracted with EtOAc (150 mL) and the combined organic phases were washed with water (2×100 mL) and then concentrated in vacuo to furnish 32.8 g of crude product as dark oil. The crude product was then purified by column chromatography. The crude product was diluted with EtOAc/toluene/hexanes 15:15:70 (w/w) and then charged onto a silica gel column; eluent EtOAc/toluene/hexanes 15:15:70 (w/w). The combined product fractions were concentrated and subsequently dissolved in EtOH (100 mL) and then filtered. The solution was concentrated to dryness, furnishing 2,2,4-trimethyl-2H-chromen-6-ol as brownish crystals (27 g, 92 wt % by qNMR, 76% yield). ¹H NMR (300 MHz, CDCl₃) δ 1.39 (s, 6H), 1.97 (d, J=1.5 Hz, 3H), 4.64 (s, 1H, OH), 5.45 (q, J=1.4 Hz, 1H), 6.58-6.63 (m, 1 H), 6.65-6.72 (m, 2H) ppm.

### Example 2: Synthesis of Compound of Formula (2) (2,2-dimethyl-2H-chromen-6-ol)

See FIG. 5:

Step 1: CuCl₂, DBU (=1,8-diazabicyclo[5.4.0]undec-7-ene), CH₃CN;

Step 2: PPh₃AuNTf₂, DCM

Step 1: A 500 mL 4-necked flask with magnetic stirrer, thermometer, and argon supply was charged with hydroquinone (22.1 g, 200 mmol, 1.0 mol equiv.) and dissolved in acetonitrile (200 mL). The solution was cooled to 0-4° C. (ice-bath) and DBU (66.5 g, 440 mmol, 2.2 mol equiv.) and copper(II) chloride (0.080 g, 0.596 mmol, 0.3 mol %) were added. Then, 3-chloro-3-methylbut-1-yne (21 g, 200 mmol, 1.0 mol equiv.) was added dropwise within 20 min. After stirring at 0-4° C. for 1.5 h the beige-brown reaction was quenched by slow addition to a vigorous stirring mixture of HCl (100 mL, 25% in water) and ice (100 g). Ethyl acetate (300 mL) was added and the water phase was extracted with ethyl acetate (200 mL). The combined organic phases were washed with HCl (100 mL, 1N in water), sat. aq. NaHCO₃ (100 mL) and finally brine (100 mL, 10% NaCl in water), dried over sodium sulfate, filtered and concentrated in vacuo (40° C./150-20 mbar). The residue was purified by column chromatography: The sample was diluted with little eluent and charged onto a silica gel column; eluent gradient heptane/EtOAc 90:10 to 80:20 (w/w). The pure fractions were combined, concentrated in vacuo (40° C./200-10 mbar) and dried under high vacuum at 40° C., furnishing 6.46 g 4-((2-methylbut-3-yn-2-yl)oxy)phenol as colorless crystals (22% yield).

Step 2: An oven-dried 250 mL flask with magnetic stirrer, thermometer and argon supply was charged with 4-((2-methylbut-3-yn-2-yl)oxy)phenol (6.4 g, 34.4 mmol, 1.0 mol equiv.) was dissolved under argon atmosphere in dry DCM (100 mL) and cooled to 0° C. Ph₃PAuNTf₂ (0.046 g, 0.062 mmol, 0.2 mol %) was added and the ice bath was removed. After 100 min, another portion Ph₃PAuNTf₂ (0.25 g, 0.337 mmol, 1.0 mol %) was added. After 4 h, the dark solution was concentrated in vacuo (40° C./500-20 mbar). The dark residue was purified by column chromatography: The sample was diluted with little eluent and charged onto a silica gel column; eluent gradient heptane/EtOAc 95:5 to 70:30 (w/w). The pure fractions were combined, concentrated in vacuo (40° C./200-10 mbar) and dried under high vacuum at 40° C., furnishing 2.85 g 2,2-Dimethyl-2H-chromen-6-ol as off-white crystals (45% yield, mp 89-90° C.). ¹H NMR (300 MHz, CDCl₃) δ 1.42 (s, 6H), 4.66 (s, 1H, OH), 5.64 (d, J=9.8 Hz, 1H), 6.26 (d, J=9.8 Hz, 1H), 6.50 (d, J=2.8 Hz, 1H), 6.59 (dd, J=8.7, 2.8 Hz, 1H), 6.67 (d, J=8.3 Hz, 1H) ppm. ¹³C NMR (75 MHz, CDCl₃) δ 27.6 (2 C), 75.8 (1 C), 112.8 (1 C), 115.4 (1 C), 116.9 (1 C), 122.1 (2 C), 131.9 (1 C), 146.7 (1 C), 149.4 (1 C) ppm.

### Example 3: Synthesis of Compound of Formula (3) (2,2,4,5,7,8-hexamethyl-2H-chromen-6-ol)

See FIG. 2:

Step 1: Bn-Br, K₂CO₃, DMF;

Step 2: 1) MeMgCl, THE; 2) aq. HCl, pTsOH;

Step 3: BCl₃, DCM.

The starting material, 6-hydroxy-2,2,5,7,8-pentamethylchroman-4-one, was prepared as published in US 2006/193797, Example 1.

Step 1: A 3-necked 250 mL round-bottom flask equipped with thermometer, reflux condenser, argon inlet, magnetic stirring and oil bath was charged with 6-hydroxy-2,2,5,7,8-pentamethylchroman-4-one (14.94 g, 63.8 mmol, 1.0 mol equiv.), K₂CO₃ (17.63 g, 127.6 mmol, 2.0 mol equiv.) and dissolved in DMF (65 mL). The apparatus was then inertized with argon. Subsequently, benzyl bromide (12.0 g, 70.2 mmol, 1.1 mol equiv.) was added and the reaction was stirred for 1 h at room temperature, then 1 h at 40° C. The mixture was cooled to room temperature and quenched slowly with water (150 mL). EtOAc (150 mL) was added. The aqueous phase was extracted with EtOAc (100 mL). The combined organic phases were washed with water (2×50 mL), then filtered and concentrated in vacuo, furnishing the crude product as brown solid. The crude product was recrystallized by dissolving in toluene (19 g) at 55° C. and slow addition of n-hexane (77 g) at 50-55° C. The solution was allowed to cool to room temperature, upon which crystals formed spontaneously at 20° C. The mixture was cooled to 0° C. for 1 h. The crystals were isolated by filtration; the filter cake was washed with the mother liquor and then with little cold n-hexane. The crystals were dried in vacuo at 60° C. for 18 h, furnishing 6-(benzyloxy)-2,2,5,7,8-pentamethylchroman-4-one as colorless solid (14.7 g, 45.3 mmol, mp 89-90° C.). A second crop of crystals was obtained by dissolving the concentrated mother liquor in toluene (4.5 g) and addition of n-hexane (19 g): 3.03 g of 6-(benzyloxy)-2,2,5,7,8-pentamethylchroman-4-one crystals could be obtained (˜9.3 mmol, mp 88-89° C.); combined yield: 85%.

Step 2: A 3-necked 250 mL round-bottom flask equipped with thermometer, reflux condenser, argon inlet, magnetic stirring and oil bath was charged with 6-(benzyloxy)-2,2,5,7,8-pentamethylchroman-4-one (16.9 g, 52.0 mmol, 1.0 mol equiv.) and dissolved in dry THF (104 mL). The apparatus was then inertized with argon. Subsequently, MeMgCl (3 M in THF, 26 mL, 78 mmol, 1.5 mol equiv.) was added dropwise within 45 min at 15-20° C. The reaction was then stirred for 4 h at room temperature. HPLC analysis indicated ˜55 area % starting material and ˜35 area % addition product. No further conversion was observed; probably due to steric constrains around the ketone moiety. The mixture was cooled to 0° C. and HCl (4 M in water, 29 mL, 116 mmol, 2.2 mol equiv.) was added slowly over 20 min at 0-10° C. p-Toluenesulfonic acid (0.33 g, 1.7 mmol, 3 mol %) was added and the mixture was heated to reflux for 30 min. After cooling, water (100 mL) and Et₂O (100 mL) were added. The aqueous phase was extracted with Et₂O (2×100 mL) and the combined organic phases were washed with water (2×50 mL). The organic phases were concentrated in vacuo (60° C./26 mbar). The residue was re-dissolved in toluene (25 mL) and EtOH (25 mL) and concentrated in vacuo (60° C./23 mbar), furnishing the crude product as yellow oil that crystallized on standing (18.24 g,). The crude product was purified by column chromatography (eluent toluene/hexane 50:50 (w/w) until complete elution of product, then toluene and finally toluene/EtOAc 95:5 (w/w) to recover starting material). Product fractions were combined, filtered and concentrated in vacuo, furnishing 6-(benzyloxy)-2,2,4,5,7,8-hexamethyl-2H-chromene in an 82:18 mixture with its olefin-isomer as colorless solid (5.46 g, 16.9 mmol, 82:18 ratio of olefin-isomers by HPLC, 33% yield).

Fractions containing starting material were combined, filtered and concentrated to furnish 12.8 g of starting material, which still contained some toluene, even after prolonged drying. ¹H NMR (major isomer, 300 MHz, CDCl₃) δ 1.35 (s, 6H), 2.14 (s, 3H), 2.18 (d, J=1.5 Hz, 3H), 2.23 (s, 3H), 2.41 (s, 3H), 4.73 (s, 2H), 5.49 (q, J=1.5 Hz, 1H), 7.31-7.46 (m, 3H), 7.46-7.55 (m, 2H) ppm.

Step 3: A 3-necked 250 mL round-bottom flask equipped with thermometer, reflux condenser, argon inlet, magnetic stirring and dry ice bath was charged with 6-(benzyloxy)-2,2,4,5,7,8-hexamethyl-2H-chromene (5.90 g, 18.3 mmol, 82:18 mixture of olefin isomers, 1.0 mol equiv.) and dissolved in dichloromethane (100 mL). The apparatus was then inertized with argon. Subsequently, the solution was cooled to −40° C. and BCl₃ (1 M in DCM, 39.5 mmol, 2.1 mol equiv.) was added dropwise over 35 min. The mixture was stirred for 4 h at −40° C. and then warmed to −20° C. Brine (11 mL) was added dropwise over 9 min, keeping the temperature between −20 and −10° C. At 0° C., water (40 mL) was added. A suspension formed, which was filtered and the cake was washed with DCM (50 mL). The aqueous phase of the filtrate (2 phases) was separated and extracted with DCM (50 mL). The combined organic phases were then washed with water (3×50 mL) and concentrated in vacuo (60° C./20 mbar), furnishing the crude product as yellow oil. The crude product was then purified by column chromatography on silica gel, eluting with hexanes/EtOAc 93:7 (w/w). The product fractions contained varying ratios of product and olefin-isomer. They were combined and concentrated in vacuo, furnishing PM-chromenol and its isomer as yellow oil (3.64 g, 86% yield, mp 66-67° C.). ¹H NMR (major isomer, 300 MHz, CDCl₃) δ 1.35 (s, 6H), 2.16 (s, 3H), 2.17 (d, J=1.5 Hz, 3H), 2.19 (s, 3H), 2.35 (s, 3 H), 4.30 (s, 1H), 5.50 (q, J=1.3 Hz, 1H) ppm.

### Example 4: Synthesis of Compound of Formula (4) (2,2,5,7,8-pentamethyl-2H-chromen-6-ol)

See FIG. 6:

Step 1: 1) NaBH₄, MeOH, THF; 2) HCl, pTsOH.

A 3-necked 250 mL round-bottom flask equipped with thermometer, reflux condenser, argon inlet, magnetic stirring and oil bath was charged with 6-hydroxy-2,2,5,7,8-pentamethylchroman-4-one (9.36 g, 40.0 mmol, 1.0 mol equiv.) and dissolved in THF (40 mL). The apparatus was then inertized with argon. Subsequently, NaBH₄ (1.51 g, 1.0 mol equiv.) was added followed by cautious addition of MeOH (3.25 mL, 2.0 mol equiv.) at room temperature. Gas evolution and exothermicity was observed. After 5 h, additional NaBH₄ (0.76 g, 0.5 mol equiv.) and MeOH (1.6 mL, 1.0 mol equiv.) were added. After 18 h, additional NaBH₄ (0.76 g, 0.5 mol equiv.) and MeOH (1.6 mL, 1.0 mol equiv.) were added. 2 h later, HCl (4 M in water, 40 mL, 160 mmol, 4 mol equiv.) was added slowly under slight cooling with a water bath. p-Toluenesulfonic acid (0.76 g, 4.0 mmol, 10 mol %) was added and heated to reflux for 1.5 h. The reaction was cooled to room temperature and quenched with water (100 mL). The aqueous phase was extracted with EtOAc (2×100 mL) and the combined organic phases were washed with water (2×50 mL). The organic phase was concentrated in vacuo, re-dissolved in EtOH (100 mL), filtered and concentrated in vacuo (60° C./20 mbar), furnishing the crude product as yellow oil. The crude product was recrystallized in toluene (9 g) and diluted with n-hexane (18 g). The solution was cooled to 0° C. for 1 h and then filtered. The cake was washed with the mother liquor and subsequently with n-hexane (10 mL). After drying (45° C./20 mbar, 2 h), the product was obtained as colorless solid (3.3 g, mp 75-77° C.). A second crop of crystals was obtained from the concentrated mother liquor (3.7 g); combined yield: 83%. ¹H NMR (300 MHz, CDCl₃) δ 1.40 (s, 6H), 2.14 (s, 3H), 2.17 (s, 3H), 2.19 (s, 3H), 4.22 (s, 1H), 5.65 (d, J=10.0 Hz, 1H), 6.52 (d, J=10.0 Hz, 1H) ppm.

### Example 5: Synthesis of Compound of Formula (5) (6-ethoxy-2,2,4-trimethyl-2H-chromene)

See FIG. 3, 2^{nd} step.

A 3-necked 250 mL round-bottom flask equipped with thermometer, reflux condenser, argon inlet, magnetic stirring and oil bath was charged with crude 2,2,4-trimethyl-2H-chromen-6-ol (13.0 g, 74 area % purity by HPLC, ˜50.6 mmol, 1.0 mol equiv.) and dissolved in DMSO (35 mL) and THF (70 mL) forming a yellow solution. The apparatus was inertized with argon. Subsequently, ethyl iodide (11 mL, 136.8 mmol, 2.7 mol equiv.) and K₂CO₃ (18.9 g, 136.8 mmol, 2.7 mol equiv.) were added. The suspension was then heated to 50° C. for 24 h and monitored by HPLC. After cooling to room temperature, water (100 mL) was added and diluted with diethyl ether (100 mL). The aqueous phase was extracted with diethyl ether (100 mL). The combined organic phases were washed with water (2×50 mL), concentrated in vacuo (50° C./70 mbar), and further dried at 50° C./4 mbar. The crude product was purified by column chromatography on silica gel, eluting with hexanes/iPr₂O 96:4 (w/w). Product fractions were combined and concentrated in vacuo to give a yellow oil, which was re-dissolved in Et₀H (100 mL), filtered and then concentrated in vacuo. The residue was again dissolved in Et₀H (100 mL) and concentrated in vacuo (60° C./20 mbar), furnishing EtO-TM-chromenol as yellowish oil (11.0 g, 91 wt % by qNMR, 45.9 mmol, 91% yield). ¹H NMR (300 MHz, CDCl₃) δ 1.39 (m, 6H), superimposed by 1.40 (t, J=7.0 Hz, 3H), 1.99 (d, J=1.5 Hz, 3H), 4.00 (q, J=7.0 Hz, 2 H), 5.45 (q, J=1.3 Hz, 1H), 6.65-6.77 (m, 3H) ppm.

### Example 6: Synthesis of Compound of Formula (7) (2H-chromen-6-ol)

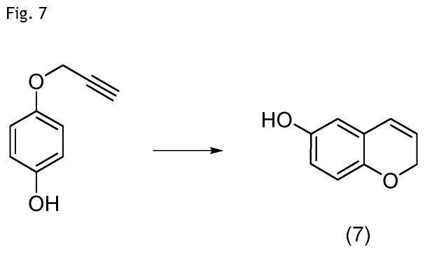

See FIG. 7:

Step 1: Ph₃PAuNTf₂, DCM.

The starting material, 4-(prop-2-yn-1-yloxy)phenol, was prepared as described in J. C. Jaen, L. D. Wise, T. G. Heffner, T. A. Pugsley, L. T. Meltzer. Dopamine autoreceptor agonists as potential antipsychotics. 2. (Aminoalkoxy)-4H-1-benzopyran-4-ones. J. Med. Chem. 1991, 34, 248-256.

A 250 mL round-bottom flask equipped with magnetic stirrer and argon supply was charged with 6.5 g 4-(prop-2-yn-1-yloxy)phenol (43.8 mmol, 1.0 mol equiv.) under argon, dissolved in dry DCM (100 mL) and cooled under stirring to 0-4 C. Subsequently, Ph₃PAuNTf₂ (0.058 g, 0.079 mmol, 0.18 mol %) was added and the reaction was stirred for 40 min at 0-4 C (cooled with an ice bath). After 2 h the dark reaction solution was concentrated in vacuo (40° C./200-10 mbar). The dark residue was purified by column chromatography: The sample was diluted with little EtOAc and charged onto a silica gel column; eluent gradient heptane/EtOAc 90:10 to 75:25 (w/w). The pure product fractions were combined, concentrated in vacuo (40° C./200-10 mbar) and dried under high vacuum at 40° C., furnishing 4.8 g 2H-chromen-6-ol as an oil (31.8 mmol, purity 98.2% by qNMR, 73% yield). ¹H NMR (300 MHz, CDCl₃) δ 4.72-4.79 (m, 2H), superimposed by 4.76 (br s, 1H, OH), 5.82 (dt, J=9.8, 3.6 Hz, 1H), 6.37 (dt, J=9.9, 1.6 Hz, 1H), 6.49 (d, J=3.0 Hz, 1H), 6.55-6.63 (m, 1H), 6.63-6.71 (m, 1H) ppm. ¹³C NMR (75 MHz, CDCl₃) δ 65.4 (1 C), 113.1 (1 C), 115.4 (1 C), 116.3 (1 C), 123.1 (1 C), 123.3 (1 C), 124.4 (1 C), 147.8 (1 C), 149.8 (1 C) ppm.

### Example 7: Antioxidant Activities in Pet Food, Poultry Meal and Fish Meal

The compounds of formulae (1) to (7) have been tested and their antioxidant efficacy values (“EV”) in pet food, poultry meal and/or fish meal determined.

Determination of the Antioxidant Efficacy Value “EV”

Oxidative stability was assessed using an Oxipres (Mikrolab Aarhus A/S, Hojbjerg, Denmark). The ML OXIPRES® is designed to monitor the oxidation of heterogeneous products. Consumption of oxygen results in a pressure drop which is measured by means of pressure transducers. The samples are heated to accelerate the process and shorten the analysis time (Mikrolab Aarhus 2012).

Sample weights were 50 g. They were loaded into the Oxipres vessels and placed inside the stainless-steel pressure vessel and sealed. The pressure vessels were purged with pure oxygen and filled to an initial oxygen pressure of 5 bar and maintained at 70° C. during measurement (D. Ying, L. Edin, L. Cheng, L. Sanguansri, M. A. Augustin, LWT-Food Science and Technology 2015, 62, 1105-1111: “Enhanced oxidative stability of extruded product containing polyunsaturated oils.”).

The oxygen pressure was recorded as function of time. After sample load and temperature rise the pressure in the device is increasing within 10 hours. Thereafter it is decreasing. Consequently, the starting pressure is considered to be the pressure after 10 hours. The analysis ends after 130 hours at 70° C. The oxygen consumption ‘02’ of the tested sample is calculated as follows:

$$O 2 consumption ( as \% ) = 1 - [ \frac{Pressure after 130 hours in Oxipres}{Pressure after 10 hours in Oxipres} ]$$

TABLE 1
Oxipres performance of the three matrices

| | Matrix 1 | Matrix 2 | Matrix 3 |
|---|---|---|---|
| | Pet food | Poultry meal | Fish meal |
| Oxipres results-O₂ consumption | 25% | 32% | 31% |
| CV (=coefficient of variation) | 19% | 13% | 9% |

A factor of protection called ‘EV’ (Efficacy Value) was developed to quantify with a relative number (relative to BHT) the antioxidant effect of the tested candidates. EV was calculated as follows:

$$E {V}_{AOX candidate} = \frac{1}{( O 2 {consumption}_{AOX candidates} / O 2 {consumption}_{BHT} )}$$

EV, being relative to BHT (3,5-di-tert-butyl-4-hydroxy-toluene) (EV=1), makes it possible to compare the antioxidant compounds in a defined feed application. Here pet food, poultry meal and fish meal have been used as feed application with the composition as given in the following Table 2.

TABLE 2

| | | Pet | Poultry | Fish |
|---|---|---|---|---|
| | | food | meal | meal |
| | | Matrix | Matrix | Matrix |
| Parameters analyzed | Amount | 1 | 2 | 3 |
| Crude Protein | 8/100 g | 24 | 56 | 68 |
| Total fat | 8/100 g | 9.8 | 19.4 | 12.4 |
| Saturated fatty acids | g/100 g Fat | 32.8 | 30.8 | 20.5 |
| Mono unsaturated fatty | g/100 g Fat | 38.1 | 43.5 | 31.0 |
| acids | | | | |
| Poly unsaturated fatty acids | g/100 g Fat | 19.6 | 17.9 | 28.3 |
| Omega 3 | g/100 g Fat | 3.76 | 0.72 | 25.9 |
| Omega 6 | g/100 g Fat | 15.8 | 17.1 | 2.36 |
| Saturated fatty acids | g/100 g | 3.21 | 5.97 | 2.55 |
| Mono unsaturated fatty | g/100 g | 3.73 | 8.42 | 3.85 |
| acids | | | | |
| Poly unsaturated fatty acids | g/100 g | 1.91 | 3.45 | 3.50 |
| Omega 3 | g/100 g | 0.367 | 0.139 | 3.22 |
| Omega 6 | g/100 g | 1.55 | 3.32 | 0.294 |
| Omega 3 + 6 | g/100 g | 1.92 | 3.46 | 3.51 |
| Unsaturated Fatty acids | g/100 g | 7.56 | 15.33 | 10.86 |
| Moisture content | % | 8.0 | 4.8 | 7.3 |
| water activity | | 0.49 | 0.42 | 0.53 |
| pH | | 7.8 | 7.6 | 7.4 |

Compounds of formulae (1) to (7) were mixed each into the matrix 1, 2 or 3 (pet food, poultry meal, fish meal) in an equimolar ratio compared to BHT. Batches of 200 g feed were produced in order to handle a minimum of 30 mg of antioxidant. First, a 1% pre-dilution of the antioxidant with the feed material was made. Then this pre-dilution was added to the final batch, mixed, sieved (1.25 mm sieve) and mixed using a turbula® mixer. Thereafter 55 g of the final batch were packed into polyethylene bags and stored at 4° C. till time of Oxipres assay. Spare sample were stored at 4° C. The results are shown in the following Tables 3-5. An efficacy value 0.7 has been considered as acceptable, an efficacy value the efficacy value of alpha-tocopherol as good, and an efficacy value the efficacy value of BHT as very good.

The results in pet food are shown in the following Table 3. Compound of formula (1) showed a higher efficacy value than alpha-tocopherol (EV=0.77).

TABLE 3

| | Compound of formula (1) |
|---|---|
| | 2,2,4-trimethyl-2H-chromen-6-ol |
| | 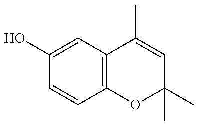 |
| EV in pet food | 0.97 |

The results in poultry meal are shown in the following Table 4. Compounds of formulae (1) and (2) showed a higher efficacy value than alpha-tocopherol (EV=0.74) and a higher efficacy value than BHT (EV=1) in poultry meal.

TABLE 4

| | Compound of formula (1) | Compound of formula (2) |
|---|---|---|
| | 2,2,4-trimethyl-2H- | 2,2-dimethyl-2H-chromen- |
| | chromen-6-ol | 6-ol |
| | 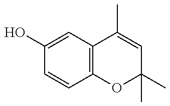 | 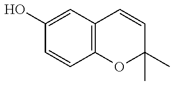 |
| EV in poultry | 1.10 | 1.11 |
| meal | | |

The results in fish meal are shown in the following Table 5. The compounds of formulae (1), (3) and (4) showed a higher efficacy value than alpha-tocopherol (EV=0.88). The compound of formula (4), even showed a higher efficacy value than BHT (EV=1) in fish meal. Though compounds of formulae (5), (6) and (7) showed an efficacy worse than alpha-tocopherol, their performance was still acceptable.

TABLE 5

| | Compound of | Compound of | Compound of |
|---|---|---|---|
| | formula (1) | formula (3) | formula (4) |
| | 2,2,4-trimethyl- | | 2,2,5,7,8- |
| | 2H-chromen-6-ol | | pentamethyl-2H- |
| | | | chromen-6-ol |
| | 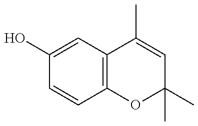 | 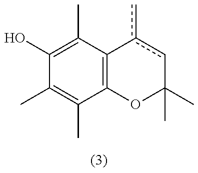 | 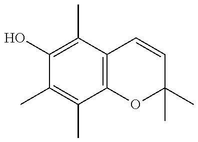 [SMILES: Cc1c(C)c2c(c(C)c1O)C=CC(C)(C)O2] |
| EV in | 0.94 | 0.89 | 1.10 |
| Fish | | | |
| meal | | | |
| | Compound of | Compound of | Compound of |
| | formula (5) | formula (6) | formula (7) |
| | 6-ethoxy-2,2,4- | 6- | 2H-chromn-6- |
| | trimethyl-2H- | hydroxycoumarin | ol |
| | chromene | | |
| | 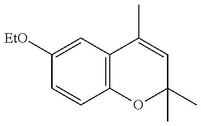 [SMILES: CCOc1ccc2c(c1)C(C)=CC(C)(C)O2] | 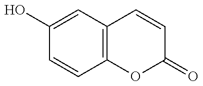 [SMILES: O=c1ccc2cc(O)ccc2o1] | 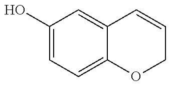 [SMILES: Oc1ccc2c(c1)C=CCO2] |
| EV in | 0.77 | 0.75 | 0.72 |
| Fish | | | |
| meal | | | |

### Example 8: Antioxidant Activities in Fish Oil and Algal Oil

Compound of formula (1) has been tested. The blank oil, i.e. oil without any antioxidant, and oil containing “MNT” have been used as benchmark. Any compound better in antioxidant activity than the blank oil indicates that it has antioxidant activity. The comparison with MNT gives an indication about the amount of the antioxidant effect, relative to the activity of MNT.

“MNT” are mixed natural tocopherols commercially available as e.g. “Tocomix 70 IP” from AOM (Buenos Aires, Argentina). Tocomix 70 IP comprises d-alpha-tocopherol, d-beta-tocopherols, d-gamma-tocopherols and d-delta-tocopherol, whereby the total amount of tocopherols is at least 70.0 weight-% and the amount of non-alpha tocopherols is at least 56.0 weight-%.

Compound of formula (1) was evaluated primarily for its oxidative stability by the Oil Stability Index measurements.

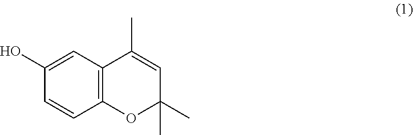

Two different levels of compound of formula (1) (0.5 and 2 mg/g) were used in 5 g of natural fish oil (Product Code: FG30TG) and used in the Oxidative Stability Instrument at 80° C. with the air flow rate of 40 psi. The solubility of different amounts and types of antioxidants used in 051 was checked before and after the application. Crude algal oil contained about 1.6 mg/g of mixed natural tocopherols (MNT) prior to use in these experiments whereas fish oil did not contain any antioxidants. Compound of formula (1) was used in the Oxidative Stability instruments under the same conditions used for fish oil evaluations. Also, the synergistic effect of compound of formula (1) with ascorbyl palmitate (AP) was determined using the OSI values. The polymers generated at the end of the experiment were determined by LC.

Results:

The solubility of the amounts of compound of formula (1) used in the oxidative stability study are shown in Table 6.

TABLE 6
Solubility of compound of formula (1) in fish oil

| | | | Solubility in fish oil | |
|---|---|---|---|---|
| | | Amount | Room | |
| Compound | Appearance | (mg/g) | temp. | 80° C. |
| compound | Yellow | 0.5 | Soluble | Soluble |
| of formula | powder | 2.0 | Soluble | Soluble |
| (1) | | 2.0 | Soluble | Soluble |

The Oil Stability Index for compound of formula (1) at 500 and 2000 ppm levels, in comparison with the same amounts of MNT, is shown in Table 7.

TABLE 7
Oxidative stability of FG30TG fish oil with compound of formula (1)

| | OSI (h) | SD |
|---|---|---|
| Blank (FG30TG) | 4.70 | 0.1 |
| 0.5 mg/g of compound of formula (1) | 5.95 | 0.2 |
| 2 mg/g of compound of formula (1) | 6.73 | 1.0 |
| 0.5 mg/g of MNT | 6.93 | 0.2 |
| 2 mg/g of MNT | 7.925 | 0.1 |

(SD = standard deviation)

The Protection Factors of compound of formula (1) in fish oil is shown in Table 8.

TABLE 8
Protection Factors of compound of formula (1) in FG30TG fish oil

| | Protection Factor |
|---|---|
| | (%) |
| 0.5 mg/g of compound of formula (1) | 25.26 |
| 2 mg/g of compound of formula (1) | 41.58 |
| 0.5 mg/g of MNT | 45.79 |
| 2 mg/g of MNT | 66.84 |

The improvement of the oxidative stability of the oil soluble compound of formula (1) when combined with AP is shown in Table 9.

TABLE 9
Improvement of the effect of compound of formula (1)
in FG30TG fish oil using AP

| | OSI (h) | SD |
|---|---|---|
| Blank (FG30TG) | 4.63 | 0.0 |
| 2 mg/g of compound of formula (1) | 5.70 | 0.4 |
| 2 mg/g of compound of formula (1) + 0.5 mg/g of AP | 7.60 | 0.1 |
| 2 mg/g of MNT | 8.03 | 0.9 |
| 2 mg/g of MNT + 0.5 mg/g of AP | 15.25 | 1.5 |

(SD = standard deviation)

Improvements of the Protection Factors of the oil soluble compound of formula (1) with AP in fish oil is shown in Table 10.

The Protection Factors of compound of formula (1) as well as MNT in fish oil could be improved by the addition of AP (see Table 10) indicating the possibility of combining AP to all the compounds of formulae (1) to (7) to improve the oxidative stability of matrices containing high amounts of unsaturated fatty acids such as fish oil.

TABLE 10
Improvement of the Protection Factors of compound of
formula (1) with AP in FG30TG fish oil

| | Protection Factor |
|---|---|
| | (%) |
| 2 mg/g of compound of formula (1) | 23.1 |
| 2 mg/g of compound of formula (1) + | 64.1 |
| 0.5 mg/g of AP | |
| 2 mg/g of MNT | 73.3 |
| 2 mg/g of MNT + 0.5 mg/g of AP | 229.4 |

Polymers generated at the end of the stabilization experiment of fish oil with compound of formula (1) and AP are shown in Table 11.

TABLE 11
Reduction of polymers in FG30TG oil with a compound (AP)
synergistic to compound of formula (1)

| | Polymers | |
|---|---|---|
| | (%) | SD |
| Blank (FG30TG) | 43.97 | 3.7 |
| 2 mg/g of compound of formula (1) | 34.67 | 3.4 |
| 2 mg/g of compound of formula (1) + 0.5 mg/g of AP | 32.65 | 1.0 |
| 2 mg/g of MNT | 33.87 | 1.1 |
| 2 mg/g of MNT + 0.5 mg/g AP | 12.72 | 2.6 |

(SD = standard deviation)

Polymers are combination of complex compounds generated at the end of the oxidation cascade of unsaturated fatty acids and, they indicate the levels of overall oxidation of the matrix. The generation of such polymers in fish oil containing compound of formula (1) could be reduced considerably when AP was added as a synergistic compound (Table 11).

Tables 12, 13 and 14 show the PV (peroxide value), p-AV (p-anisidine value) and CD (conjugated dienoic acid in %) of the fish oil samples stabilized with compound of formula (1). For the storage stability study compound of formula (1) was used in fish oil at only 2 mg/g level. Compared to the same level of MNT, compound of formula (1) showed much higher PVs than those of MNT (Table 12). MNT had the lowest PV values. There was no considerable variation in p-AV and CD (Tables 13-14) during the storage.

TABLE 12
Variation of PV with compound of formula (1) in FG30TG

| | | 4 | 6 | 8 | 11 | 13 | 17 |
|---|---|---|---|---|---|---|---|
| | Initial | days | days | days | days | days | days |
| Blank(FG30TG) | 0.9 | 1.6 | 2.2 | 2.7 | 5.6 | 7.8 | 11.9 |
| 2 mg/g of MNT | 0.9 | 1.1 | 1.2 | 1.4 | 1.7 | 1.5 | 2.1 |
| 2 mg/g of | 0.9 | 2 | 2.6 | 3.8 | 6.8 | 9.8 | 12.1 |
| compound of | | | | | | | |
| formula (1) | | | | | | | |

TABLE 13
Variation of p-AV with compound of formula (1) in FG30TG

| | | 4 | 6 | 8 | 11 | 13 | 17 |
|---|---|---|---|---|---|---|---|
| | Initial | days | days | days | days | days | days |
| Blank(FG30TG) | 9.9 | 9.8 | 9.9 | 10.5 | 10.9 | 9.9 | 9.8 |
| 2 mg/g of MNT | 9.9 | 10.3 | 9.9 | 10.1 | 10 | 9.8 | 10 |
| 2 mg/g of | 9.9 | 4.4 | 7.2 | 6.8 | 7.6 | 8.1 | 8.5 |
| compound of | | | | | | | |
| formula (1) | | | | | | | |

TABLE 14
Variation of CD with compound of formula (1) in FG30TG

| | | 4 | 6 | 8 | 11 | 13 | 15 |
|---|---|---|---|---|---|---|---|
| | Initial | days | days | days | days | days | days |
| Blank(FG30TG) | 0.7 | 0.7 | 0.6 | 0.7 | 0.7 | 0.8 | 0.7 |
| 2 mg/g of MNT | 0.7 | 0.7 | 0.6 | 0.7 | 0.7 | 0.7 | 0.7 |
| 2 mg/g of | 0.7 | 0.8 | 0.8 | 0.9 | 0.9 | 0.8 | 0.8 |
| compound of | | | | | | | |
| formula (1) | | | | | | | |

The Oil Stability Indices of compound of formula (1) in crude algal oil are shown in Table 15. This algal oil already contained about 1.6 mg/g of MNT. Addition of extra 2 mg/g of MNT to algal oil did not improve the 051 values either (Table 15).

TABLE 15
Oxidative stability of crude algal oil with compound of formula (1)

| | OSI (h) | SD |
|---|---|---|
| Crude algal oil | 14.85 | 0.4 |
| 0.5 mg/g of compound of formula (1) | 16.60 | 0.6 |
| 2 mg/g of compound of formula (1) | 18.55 | 1.0 |
| 0.5 mg/g of MNT | 15.95 | 0.1 |
| 2 mg/g of MNT | 16.35 | 0.2 |

(SD = standard deviation)

Results

Compound of formula (1) shows antioxidant properties in fish oil and algal oil at different levels.

1-22. (canceled)

23. Use of a compound of formula (I) and/or a compound of formula (II) as antioxidant in feed ingredients and/or feed, 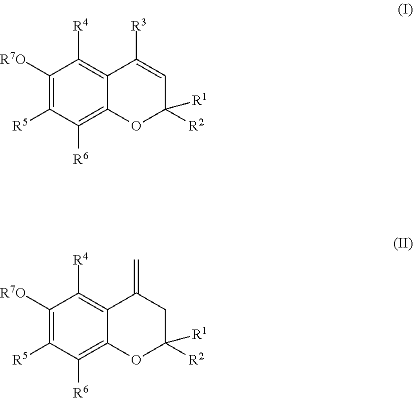
wherein R¹ and R² are independently from each other H or C₁₋₁₁-alkyl or (CH₂)ₙ—OH with n being an integer from 1 to 6 or R¹ and R² together represent a keto group, and wherein R³, R⁴, R⁵, and R⁶ are independently from each other H or C₁₋₆-alkyl or C₁₋₆-alkoxy, and R⁷ is H or C₁₋₆-alkyl.

24. The use according to claim 23, whereby in compound of formula (I) and/or in compound of formula (II) R¹ and R² are independently from each other H or C₁₋₄-alkyl or (CH₂)ₙ—OH with n being an integer from 1 to 4, R³, R⁴, R⁶ and R⁷ are independently from each other H or C₁₋₄-alkyl, and R⁵ is H or C₁₋₄-alkyl or C₁₋₄ -alkoxy, preferably whereby in compound of formula (I) and/or in compound of formula (II) R¹ and R² are independently from each other H or C₁₋₂ -alkyl or (CH₂)ₙ—OH with n being 1 or 2, R³, R⁴, R⁶ and R⁷ are independently from each other H or C₁₋₂ -alkyl, and R⁵ is H or C₁₋₂ -alkyl or C₁₋₂ -alkoxy, more preferably whereby in compound of formula (I) and/or in compound of formula (II) R¹ and R² are independently from each other H or methyl or (CH₂)—OH, R³, R⁴, R⁶ and R⁷ are independently from each other H or methyl or ethyl, and R⁵ is H or methyl or methoxy.

25. The use according claim 23, whereby the compound of formulae (I) or (II) is one of the following compounds of formulae (1) to (7):

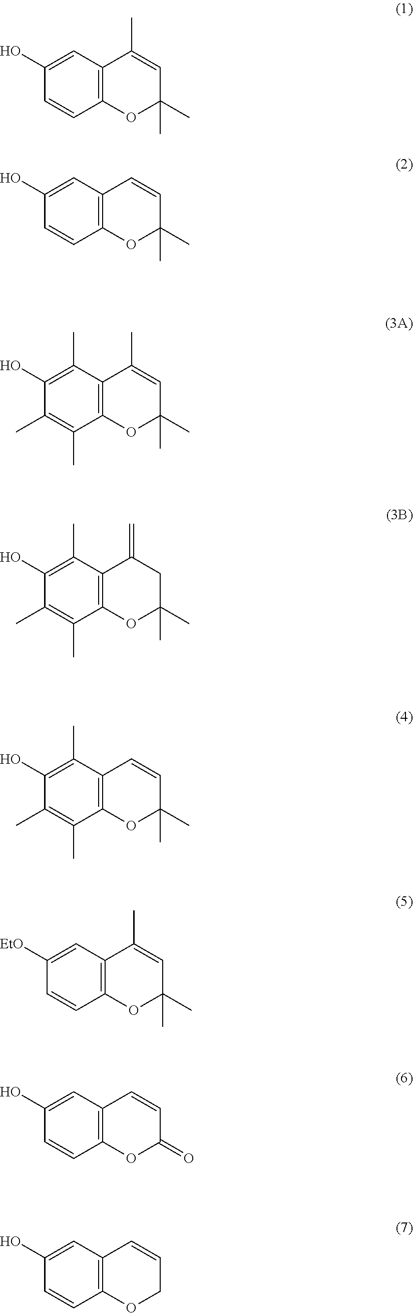

26. The use according to claim 23, whereby the feed ingredient is either poultry meal or fish meal or insect meal or a PUFA-containing oil, wherein the PUFA-containing oil is preferably marine oil or microbial oil or fungal oil or algal oil or PUFA-containing plant oil, preferably wherein the PUFA-containing oil is marine oil or algal oil, more preferably wherein the PUFA-containing oil is algal oil.

27. Feed ingredient comprising at least one compound of formula (I) and/or at least one compound of formula (II), 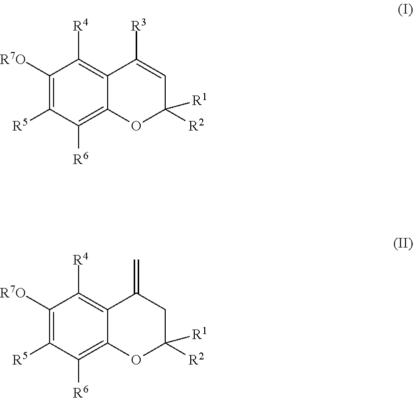
wherein R¹ and R² are independently from each other H or C₁₋₁₁-alkyl or (CH₂)ₙ—OH with n being an integer from 1 to 6 or R¹ and R² together represent a keto group, and wherein R³, R⁴, R⁵, and R⁶ are independently from each other H or C₁₋₆-alkyl or C₁₋₆-alkoxy, and R⁷ is H or C₁₋₆-alkyl.

28. The feed ingredient according to claim 27, whereby the feed ingredient is either poultry meal or fish meal or insect meal or a PUFA-containing oil, wherein the PUFA-containing oil is preferably marine oil or microbial oil or fungal oil or algal oil or PUFA-containing plant oil, preferably wherein the PUFA-containing oil is marine oil or algal oil, more preferably wherein the PUFA-containing oil is algal oil.

29. The feed ingredient according to claim 27 additionally comprising a mixture of 2-tert-butyl-4-methoxyphenol and 3-tert-butyl-4-methoxyphenol and/or additionally comprising esters of ascorbic acid with linear C₁₂₋₂₀ alkanols, preferably esters of ascorbic acid with linear C₁₄₋₁₈ alkanols, more preferably ascorbyl palmitate and/or additionally comprising alpha-tocopherol and/or gamma-tocopherol.

30. The use according to claim 23, whereby the feed is feed for aquatic animals, feed for terrestrial animals (preferably feed for poultry, pets or pigs) or feed for insects.

31. Feed for aquatic animals or terrestrial animals or insects comprising at least one compound of formula (I) and/or at least one compound of formula (II), 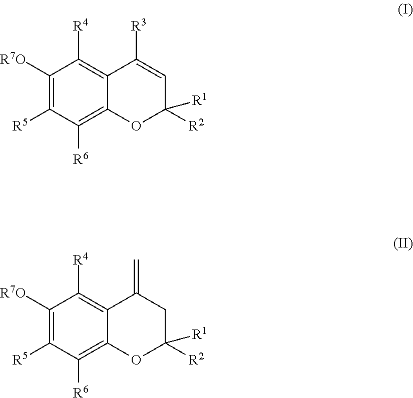
wherein R¹ and R² are independently from each other H or C₁₋₁₁-alkyl or (CH₂)ₙ—OH with n being an integer from 1 to 6 or R¹ and R² together represent a keto group, and wherein R³, R⁴, R⁵, and R⁶ are independently from each other H or C₁₋₆-alkyl or C₁₋₆-alkoxy, and R⁷ is H or C₁₋₆-alkyl.

32. The feed according to claim 31 being pet food, feed for poultry or feed for pigs.

33. 2,2,4,5,7,8-hexamethyl-2H-chromen-6-ol of formula (3).

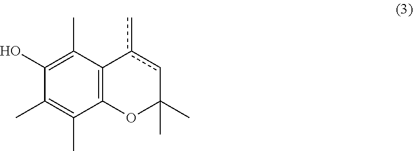

34. A process for the manufacture of a mixture of compounds of formulae (3a) and (3b) comprising the following steps: 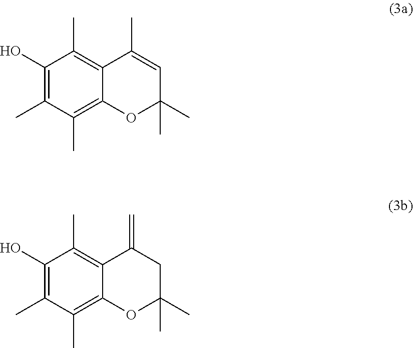 [SMILES: CC1=CC(C)(C)Oc2c(C)c(C)c(O)c(C)c21 ; C=C1CC(C)(C)Oc2c(C)c(C)c(O)c(C)c21]
a) optional protection of the hydroxy group of compound of formula (I) to obtain the compound of formula (II), 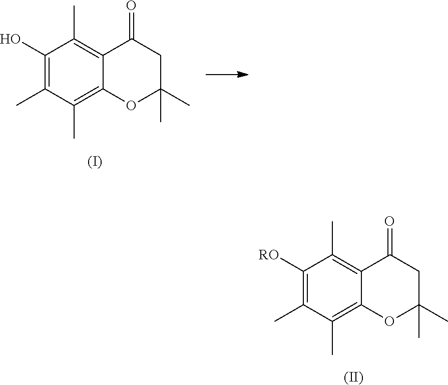
and
b) methyl-Grignard addition to the compound of formula (II) and water elimination to obtain a mixture of compounds of formulae (IIIa) and (IIIb); or methyl-Grignard addition to the compound of formula (I) and water elimination to obtain a mixture of compounds of formulae (3a) and (3b), respectively; 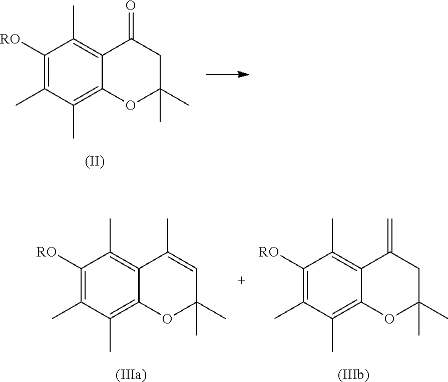
and
c) optional deprotection of the ether compounds of formulae (IIIa) and (IIIb) to a mixture of compounds of formulae (3a) and (3b)

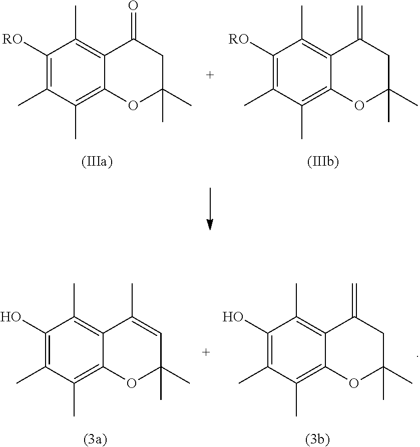

35. 6-ethoxy-2,2,4-trimethyl-2H-chromene of formula (5) with Et being ethyl.

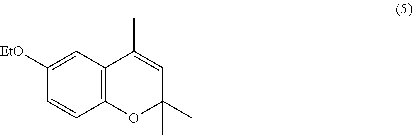 [SMILES: CCOc1ccc2c(c1)C(C)=CC(C)(C)O2]

36. A process for the manufacture of compound of formula (5) with Et being ethyl comprising the following steps: 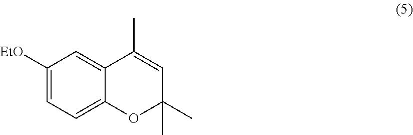 [SMILES: CCOc1ccc2c(c1)C(C)=CC(C)(C)O2]
i) methyl-Grignard addition to compound of formula (IV) and water elimination to obtain the compound of formula (1), 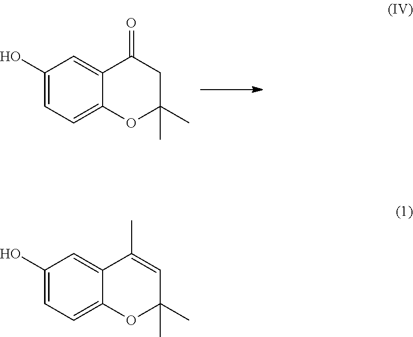
and
ii) etherification of compound of formula (1) to obtain the compound of formula (5) 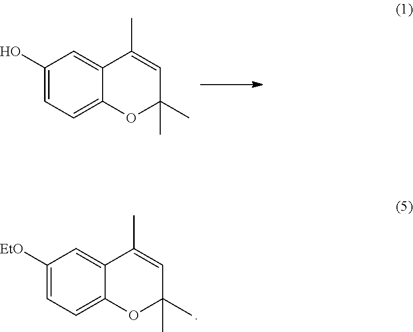 [SMILES: CC1=CC(C)(C)Oc2ccc(O)cc21 ; CCOc1ccc2c(c1)C(C)=CC(C)(C)O2]
